SEQUENCE LISTING (13 sequences)

SEQ ID NO: 1 - DNA, 1482 bp, Bacteroides thetaiotaomicron
  misc_feature 1..1482: note = Bacteroides thetaiotaomicron strain NCIMB 42341
  source 1..1482: mol_type = other DNA
cttttacaatgaagagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggtataatcagaccgcatggtttgattattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggc

SEQ ID NO: 2 - DNA, 1475 bp, Bacteroides thetaiotaomicron
  source 1..1475: mol_type = other DNA
cantgaagagtttgatcctggctcaggatnaacgctagctacaggcttaacacatgcaagtcgaggggcagcatttcnntttgcttgcaaactnnagatggcgaccggcgcacgggtgagtaacacgtatccaacctgccgataactcggggatagcctttcgaaagaaagattaatacccgatggcataatcanaccgcatggtcttattattaaagaatttcggttatcgatggggatgcgttccattaggcagttggtgaggtaacggctcacnaaaccttcgatggataggggttctgagaggaaggtcccccacattggaactgagacacggtccnaactcctacgggaggcagcagtgaggaatattggtcaatgggcgcaggcctnaaccagccaagtagcgtgaaggatgactgccctatgggttgtaaactnctnttatatgggaataaagtnttccacgtgtggaattttgtatgtaccatatgaataaggatcggctaactccgtgccagcagccgcggtnatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctnatcccaaaagcctctctcagttcggatcgaagtctgcaacccgacttcgtgaagctggattcgctagtaatcgcgcatcagccatggcgcggtgaatacgttcccgggccttgtacacaccgcccgtcaanccatganagccgggggtacctgaagtacgtaaccgcaaggagcgtcctagggtaaaactggtaattgggg

SEQ ID NO: 3 - DNA, 1482 bp, Bacteroides thetaiotaomicron
  source 1..1482: mol_type = other DNA
cttntacaatgaagagtttgatcctggctcaggatnaacgctagctacaggcttaacacatgcaagtcnaggggcagcatttcagtttgcttgcaaactggagatggcgaccggcgcacgggtgagtaacacgtatccaacctgccgataactcggggatagcctttcgaaagaaagattaatacccnatggtataatcagaccgcatngtcttrttattaaagaatttcggttatcgatggggatgcgttccattaggcagttggtgaggtaacggctcacnnaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccncgntnatacggagnatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggccngcacaagcggaggaacatgtggtttaattcgatgatacgcgaggaaccttacccgggcttaaattgcatttgaatatattggaaacagtatagccgyaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacngcccntacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggcnttgtacacaccgcccgtcaagccatgaaagccgggggtacctgaagtacgtaaccgcaaggagcgtcctagggtaaaactggtaattggggc

SEQ ID NO: 4 - DNA, 1416 bp, Bacteroides thetaiotaomicron
  source 1..1416: mol_type = other DNA
gttttccctaggacgctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttacggctatactgtttccaatatattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccantgtgggggaccttcctctcagaacccctatccatcgaaggtttggtgagccgttacctcaccaactgcctaatggaacgcatccccatcgataaccgaaattctttaataacaagaccatgcggtctaattataccatcggatattaatctttctttcgaaaggctatccccgagttatcggcaggttggatacgtgttactcacccgtgcgccggtcgccatcttcagttgcaagcaaactgaaatgctgcccctcgacttgcatggtaagcc

SEQ ID NO: 5 - DNA, 1393 bp, Bacteroides thetaiotaomicron
  misc_feature 1..1393: note = Bacteroides thetaiotaomicron BT-B
  source 1..1393: mol_type = other DNA
gctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcataacctgttagtaactaaagataagggttgcgctcgttatggcacttaagccgacacctcacggcacgagctgacgacaaccatgcagcaccttcacatttgccttgcgactaacctgtttccagattattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctaattataccatcgggtattaatctttctttcgaaaggctatccccgagttatcggcaggttggatacgtgttactcacccgtgcgccggtcgccatctccagtttgcaagcaaactgaaatgctgcccctcgactgca

SEQ ID NO: 6 - DNA, 1393 bp, Bacteroides thetaiotaomicron
  misc_feature 1..1393: note = Bacteroides thetaiotaomicron BT-C
  source 1..1393: mol_type = other DNA
gctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttacggctatactgtttccagtatattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctgattataccatcgggtattaatctttctttcgaaaggctatccccgagttatcggcaggttggatacgtgttactcacccgtgcgccggtcgccatctccagtttgcaagcaaactgaaatgctgcccctcgactgca

SEQ ID NO: 7 - DNA, 1394 bp, Bacteroides thetaiotaomicron
  misc_feature 1..1394: note = Bacteroides thetaiotaomicron BT-D
  source 1..1394: mol_type = other DNA
gctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttacggctatactgtttccagtatattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctgattataccatcgggtattaatctttctttcgaaaggctatccccgagttatcggcaggtggatacgtgttactcacccgtgcgccggtcgccatctccagtttgcaagcaaactgaaatgctgcccctcgactgcatg

SEQ ID NO: 8 - DNA, 1395 bp, Bacteroides thetaiotaomicron
  misc_feature 1..1395: note = Bacteroides thetaiotaomicron BT-E
  source 1..1395: mol_type = other DNA
gctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttacggctatactgtttccagtatattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctgattataccatcgggtattaatctttctttcgaaaggctatccccgagttatcggcaggttggatacgtgttactcacccgtgcgccggtcgccatctccagtttgcaagcaaactgaaatgctgcccctcgactgcatg

SEQ ID NO: 9 - DNA, 1395 bp, Bacteroides thetaiotaomicron
  misc_feature 1..1395: note = Bacteroides thetaiotaomicron BT-F
  source 1..1395: mol_type = other DNA
gctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttacggctatactgtttccagtatattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctgattataccatcgggtattaatctttctttcgaaaggctatccccgagttatcggcaggtaggatacgtgttactcacccgtgcgccggtcgccatctccagtttgcaagcaaactgaaatgctgcccctcgactgcatg

SEQ ID NO: 10 - DNA, 1412 bp, Bacteroides thetaiotaomicron
  misc_feature 1..1412: note = Bacteroides thetaiotaomicron BT-G
  source 1..1412: mol_type = other DNA
tttactaggacgctcttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttacggctatactgtttccagtatattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctgattataccatcgggtattaatctttctttcgaaaggctatccccgagttatcggcaggttggatacgtgttactcacccgtgcgccggtcgccatctccagttgcaagcaaactgaaatgctgcccctcgactgcatgtgtagccg

SEQ ID NO: 11 - DNA, 1398 bp, Bacteroides thetaiotaomicron
  source 1..1398: mol_type = other DNA
ggacgctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttacggctatactgtttccagtatattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggncacggagttagccgatccttattcatatggtacatacaaaattccacacgtggaaaactttattcccatataaaagaagtttacaacccatagggcagtcatccttcacgctacttggctggttcaggcctgcgcccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctgattataccatcgggtattaatctttctttcgaaaggctatccccgagttatcggcaggttggatacgtgttactcacccgtgcgccggtcgccatctccagtttgcaagcaaactgaaatgctgcccctcgactgca

SEQ ID NO: 12 - DNA, 1393 bp, Bacteroides thetaiotaomicron
  source 1..1393: mol_type = other DNA
gctccttgcggttacgtacttcaggtacccccggctttcatggcttgacgggcggtgtgtacaaggcccgggaacgtattcaccgcgccatggctgatgcgcgattactagcgaatccagcttcacgaagtcgggttgcagacttcgatccgaactgagagaggcttttgggattagcatcctgtcaccaggtagctgccttctgtaccccccattgtaacacgtgtgtagccccggacgtaagggccgtgctgatttgacgtcatccccaccttcctcacatcttacgacggcagtctctctagagtcctcagcatgacctgttagtaactaaagataagggttgcgctcgttatggcacttaagccgacacctcacggcacgagctgacgacaaccatgcagcaccttcacatttgccttgcggctaacctgtttccagawtattcaaatgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagacagccagtatcaactgcaattttacggttgagccgcaaactttcacaactgacttaactgtccacctacgctccctttaaacccaataaatccggataacgctcggatcctccgtattaccgcggctgctggcacggagttagccgatccttattcatatggtacatacaaaattccacacgtggaaaactttattcccatataaaagaagtttacaacccatagggcagtcatccttcacgctacttggctggttcaggctttcgtccattgaccaatattcctcactgctgcctcccgtaggagtttggaccgtgtctcagttccaatgtgggggaccttcctctcagaacccctatccatcgaaggtttggtgagccgttacctcaccaactgcctaatggaacgcatccccatcgataaccgaaattctttaataacaagaccatgcggtctaattataccatcgggtattaatctttctttcgaaaggctatccccgagttatcggcaggttggatacgtgttactcacccgtgcgccggtcgccatctccagtttgcaagcaaactgaaatgctgcccctcgactgca

SEQ ID NO: 13 - DNA, 33038 bp, Bacteroides thetaiotaomicron
  misc_feature 1..33038: note = Bacteroides thetaiotaomicron VPI-5482
  source 1..33038: mol_type = other DNA
atgaacaaaccgaagattatacagattattgatgttgtttccaatgcgatagacggaaaccgtatcgacgaggattttataaagagttgtatttacggcaaagtgaatgcggaactgtatgcgcatcttttaggcaaatacagggggtatgacggggatttctttcagttttatttggggacggacgatcggataaaccgggctttgctggagaacctggggattaaggtcgaacccgacaaatatccggactatgatagccggattgttgcgcaggtcgtccagggaaagaaaaggtttgatatttatcctttcgagttggaagcctttaacagatatgccatgtttggcaataataatgcgctttcatgcctgaaagggatttccccgacagccgggcagaccgtccgggaaaacggtattaacgagtatggaaacgccttgaattggtcgctgttctggataaaggcaaatccggaggacaaagccctgttagtggatcacgttttgaatatccccgaacgatgatagcggaatatttcatttaccggagaaagggggataaagagccgtttatctccctgggggaaatgccgcaatacgggttaaggccgaaacagaagtttaccgggaaaaaacttaaaatagaggtgatccgcaggctttccggtgtggaaatcgagcagacggccaccactccgcagataaacgcctatatcgaggcgaatatctatgataccgaacgttggccggagtaccggaagttataccgacaggtggccggagaggtggaaaccgtggccgacatctttacgctccagtatattttagtggccgaactggaggatcagaccaggacgggaaaggactgccagccgcagccgacagatccgaaagacgagaggctgatacacctaatccggtgcgagcttatgggtgaacccctggaaatgtacaagacgatgataaacccgatcatcgccttaaagaaacgtttcgtttgatactacatctactaaaaatagtagatactacttttagtattttagttcgtacgctcaatcctgggagaaaaactaatcgggatattcattgagttcccggtatgccttagctaactcttcttgggtgtgacgtttgaactttttacggcttatgatgaaaggcaacctctgattcatttcattctcgtactcgtccaggagatgtttaccccttccggtaaggacgacatgggtacggtcgtcggaaaagcggatctcttttaaatcttccattgtatctaatatacggaggatgattttctcgtaaatgggggtacgcagtaacgggtgtgcgagcatgggggaattgtattttatatcgaaattctcgatgatctccgtctgcacttcctctatatgcttgggaagtgcttttcccagcgtgtagtaaacagctatctgaaagtctttgtaatttggaaacatgggcgtatatggtttattgggaacgtggatatttttaatatttccgttccgggattcttcggccgaaagataaaaaaatctctcgaataatctttgggatagatgcgctttttttagtaaatactaaaaatagtatttttgtaatgcgcattacagtagtgtattttgtaaacgtaacgtgtacttattcaagtattttagtaaaatgggtatcttttatatccggttatactaatattagtattttagtatttttataatgtacattacaaaaatatccgtatctttgcattcagattgaaaaacaaaaggcttatgataacgacgatccagtttaagacgttggacgacttgtatcagttttacaacgacgcaatttttaacggtgagctttcggaatgtatcgtgaatatgtcccgtcacggtggggcgttcggctttttcgccgccaaccgttggagaggggacgggcaggaaaaaaaggtcgttcacgagatcagcatcaaccccgattttatgaaccgggaagatcgggactggcattctaccctggtacatgaaatgtgccatttgtggcaggaggatttcggcaggccgagccggggagggtatcataattcgcaatgggctgacaagatgatccaggtcggactgatgccgtccgacaccggggaggccggaggcaaaaggacaggccagagcattacgcattatatcattccgggagggaagttcgagcaggttttcaatacgctaagccgggaggatctgcaaaacttgcggcttaggtacaagccgactttggcggccgtacctacccggccgatccgcatcgccggaagcgacggggacgaaacggaagaaccggaagatccggacgagggggagagcaaaagcggaaagcggaagaaatacacctgtgggtgcggctgtaacgtgtggggaaaatccgggctggtgttaaggtgcggactgtgcgacactgattttacggaacaataggaaacagtgcaaaggtcagaagcccccagtaaaaaccccgccggaggcagatttcaagcatactcgttttactaaaatactaagataagtctatactaatcttagtattttagtattattgtaatgtgcattacaatataggggtatatgaaaaagtggggatgtcgtaatacgtcaattcgccccacttttaaggcttgacgtgcaatagccgtcagcccccagacggacgggaaccaccccggccgcacacaaatatagaaaatcttccggatttaaccaaacaggtcgggatctttctttttccccctggccttgataccgaaatcctcacgcagcatctttgccagtgaattttgtgcgtgggctgtactcatttcaccgctgcaaaccttatcgtcgtaatatgcacaacggtcgtacacgtctttcagccgtccgctttccaggatcttgtcgagtgcccttaccggggtgttgtcgctcggatagcttaaagcaccgtaaacccaacggtaaacgaacgagtattgttgttgctggtcgcctcgcagttcttggggatttctcgcatggatgaacaggataatcgtgtcggctttcggtttttgcccctttttaggcgttttgcagatgaatttgtaatcgaaccatacatcggcctcgctttccttgattcgtgtctgtgcaggccttaaaaccttttggtcgatctgtgcattcgagtaggaggccgggatctccaggtctttgcagaattggtcgatcgagtaataatactcccgtctgtcccgttggctgcaaatgatcttgtagagccgcttggtgtaattgccccggagggttaaggcgatgttcttcccgtaccttgtaccgccgacacccgtaccgtagtagagcaggaaagggatcgcccaggggttgagcgtcagggctacccggtttgtgcctttgatgttgtgtaccgtcgttatcaccaccccgaacgtttccaccgtctggtggatattgggatgcacccagcgaaaggaaaagtttttccgcatcatggcgatagcctctttcagaactttgctcttgttgttcttccccccggcttcgtcgcatttgatctcgacgacaggctggttaaagaggtctttaggcagttgtttttccctggtcatgtgttgctggatctggtcgcttatgagcgtcaggaggttttcttgccactcggtcacagagtactggccgaatgttacgttattcggctggattaacattacttcttcctccgtaggaggaatatagagggtgatgtcgttggtcgccataggcttttttttttgagttcgggacaaagttaaagaaataaaagataccgccaaaaaataccaagtactttttattgtaacctttagtaaaatgggtatttattggagggggtaattttagtaaaataagtattttcccccttcggattttagtaaaatgagtattttaggttgtgtcttttcacctataacgatttgtatttcagttaattataaatgaaaaaacgctttcttacaaagaacctctacaaagactattctcacaaagaaggaaaatcttagggaatcacaggcttcaaaaacggctggaaaatgtgcattttgaaaacggaaaatgagaaacggagaaaaaatgagcgaagcgtttctttagtgcgatttttccgcgcgttaaaatgtgtaaaattttgtttttgtgttgtaaaatataaggtgatcggaacgattgccgccgttttacaaatgtacacttttttggggggcggtgtacatttgtaatgtgcattttgggggctgggtgtctgatgtattggagcgaagccgtgcggagcgttccaggagcgacgagcgaagcaaggagagagagaacgacggtttaccaaagtgagcaagcagtcattggacttagtccaatgctgattttccaaatcactgatatataactgtttacttgtttatatgtatgcgaattgcatacatattgcatacattattttgataattcattgaattatagtatattgtagccgatatttatgtatgcaaacccctatacgtgaaattatggcaacaacaagtaaagatacgtctatccgtataaaggagagtacaaggtttaggcttgatatgctgaaaggaaacaaatcgcacgatgcgttcgtggccgaaatgttactctattttgaaacaacggggatcaccccgcagtcgaatgtaatgccgccgaacattgcggccaaagagcaggcaagccgggtgatcgaagttgtccggggcattgagaaatccactaacgtaaggctgaaaaacatcgaacaactccttttgtcgcttgtcggggaagtgaaaacgccgggggacaatccggacgaatatatgcacatctcacaggtacaggaacttttggagcgttccaagcaactggaacaggaagccagggaaaaccgggagaaggccgggaaactgcaaacggatctcgaaatagcccggcaggagaaaggaactccggccgtcggatgcaatacgcacaagatcctggagatcgtggagcgtatcgacgaggtgaagaagataccgacattcaacgatacggtttatgagatagaccgtaatacgctggatatgtgggtgaaaaggttaaaggacgaactaaagagataagcctatgtttgccaaagtacacccggccgaggacgtgaaaagcggaaatacgggaagctgtcacgacctggtgcgttatctggaaaaggaaacgggggagggacagcgttttttctcccatacggagcaggatatttcacccgaacgggtaattatggatatagacgggaacaaaaaggcgttaggggctaacgacgctaaattcttcatgctttcgctcaatccgtcgcagtcggaacaaatgcaccttatcggccggaaagtggacgattttaaggaacttacaccacaggagaaaaaagaggttttccaaaagctggaggcgttcacccgttcggctatggatgaatacgccttgaacttcggacgggacaatattaggggaggccaggatctcatgtattacgcacgtgtggaaacggaacgatcctatcatccggaagatgaagaggtgaaacaggggatcgccaggatcggagagcctaaacccggtctgaacttacacgttcacgtaatcgtttcacgcaagagcctggacgggaaagtgaaactttctccgggggctaaatcggccgggaacacctgggaactggagggcagggggacggtgaaacggggtttttcacatgagggctggaaagtcagggtacaggaatgttttaaccggaaattcgattaccaggccaaagagggggaaacttatgtacgtccgcaggtatcggccgagatcgggaagatcacgaacccggagcttaaaaggatattgcaggacgagcagtttacggcggcaaaccagatcgtcgcagccatgagggaacaaggttacacgcaccaggtaaggaagggggtgcattccttttcacgggagggtgaagtgttccaggttgaacacaggctcttgaaagcgttcgaacaacctctgtctgacgagcagctaaaaagcataacggaacgtttcgacttgacaaagtacgaggcgaacccggccgggtacagggagaacggtttgcaggtaaaggatataagtttctcgacatatgtaaaagatgaagcacagaagggggaaaaggccttgaaagaggtcgcttataaagtggtttatgatgaacaaaaccatacgacggtatcgttcgccaccgtccggcagttcgcatacgagcatcaaataaacctggtgaaaagcgaaccgacggccgaagccgtcttgaagaagatcaagaacccggagcttaaaaacctgttggagaactaccgttttacgtccgctaaccagattgtcgtagccatgaaagaacaaggctacacgcataaggtacggaaaggggtacattccttttcacgggagggtgaacgggtgagcatacggcacaaggatctgaaaaaattcgccgatccgaaactggagagccgccacatggagggcattatcgaacggtttaacctgtacaagtacaagcaggagggcgtggcctaccgggaaaacggcctggaagccaagaatatttctttcctgacataccagaaagtgccgatcgagccggaagaggataaaagcgtgaccggaaagccggaaacaaaggaggaagcgcagcagccacagcaggaaacgccggggagcgaggcacatccggaagaaacggaaaacgaagcggaaacgcccatgcctgaacccgatccggtgaaatacaggaaagagcttaaagaggtgtcttatgatgtactgtttgatagggaaacgaaaacctatgtgccgatttcggcgatccggaaatacgcatacgagaatgagatcaatctgatagaccgatacaagcatgggtacgcagtcaggaacgaggatcttagggagtgcctggcaaatcccgaataccggacggtaaggcagataaacaaggccatgagggagaggggatatacgatcgaaagggacgaggccggaaactatacctacatcaagggggaaagttctttctttatggaacgccgggatttgcttgcctttaccgggtacgccaaagataccggaggccgggaaaggggaacgcaccgaagcgcagacaagacggtcgggtttatcggtggtaaggccaaacaaaagctgataaacgagatcctgggggatagcttccggacggaaaggatgcttgtaggcaacgtgaaaaaggccgtcagccttatacagaacccggctaatataaaaatgatgctgataaaacagatcggcagttttctaaacccgtttaaagagttatgatatgatgatgtatattattatagggatcgcctgcctggtggggatcatcgttgttgtgctgttgcttccaaattccgggaaacagcagcaaaaaggacagaaataccgttttgaactgtcggccgggggagggaggaaaataacctttgccgatccgttcgataatttcctggtctatggtggggcgaactccggaaagaccaaaagcataggaaagcccttgttaagccagtatatacaggccgggtttgccggatttgtctataattacaaggatttcgatctggcaaggacagccgtacacctggtaaaaaaacataattacccttatgggtgcttccagatcagtttcacggatatggaacggacgcaccggacaaatccgatacgtccttccgtggtgaagaatgaaaccttgttcttgcagctaatggatgatatgctgaccgcctaccagggaaaggacggcaaacgggacgagtggtttaacggggcgttggggatattaaggggcgtttcgatccggttttataacgattatccgcagttttgcacgataccccatatcgttaatttcatttgttcggccggaactgtccggataacgtcgttcctggagggcaagcaccagagccgggtactggccggggcattcctggacgcaaaggacagccccaagacgcaaagcagctatctaagcagccttaccaacagtctaagcacgctcgccaacgaaaaaaaggtgtgttatgtcctttccggaaatgatttcgactttaacctgatagatccggaatgtccgaaattggttgtcgtatcgaacgcctatcagatcgaaaacctgatttccccggttatatcgctcatgctttccatttcgtcccggcgtttcacgctggcgaacaaagtgccgttcttctactttctggatgaagcgaccacgttccggatcgccgatttcgagaaactgccgtccgttctaagggaatacctttgcagtttcgttttcctcacgcagtcggcggcgaagatcgagaagatatacgggaaatatgaccgcagttcgatagaatccaatttcgggaaccagttctttggccggacaaaggacatagaggccttgaaaagctacccgcttgtttttggaaaagaggagaggcagagggtttcaaagaccaccggaagcagccggggaggggagaaccgcagccgtacagtttccacgcagaaagaggaaatatacgacacgaatttctttaccagcctgaaatcgggcgagttcgtggggagtgcggcacattccaacatgaggaactttcatttgaggtttgaaatgtacgaggacaaggaagatccgcttccgatcgtgcatcccgtactggcctcggatatagaggaaaattaccagcagatcataagggatatacaaggcattgaataacaagggaagtgtggcgacattctacaaagtgtgggacaactccacacttttttgtggaatataaaaaaggggtaaaattataaaagtttgataattagtgtatttattaaaacagtagtgtattatgtcaaattggaatggattttgtacaatataattgtataaatacactccagtttgaaactccagtgcaatagcaaaggttgtttttggattggccggataagaatttatttacttaaatacttaaaaataaactccttatgaaaacgcctttgtttatcttgctccaggctactggaggtatcagaaatgaagtaaacacattccttagtgattatgccgttccggtgatcgccatgctcctgatcgtgggagtggggatcggtgtggtgatgaattatgataagattatagaccgggacggacagggaacaagaaaagagggtattgtaaacttactctgggtagttggttacatcatcatcgggcttgcgatcattgcagccgttatcgcacttatcaatagcaaacttaaaatgtccctctaatggacttccaggttagaaaggggctggaaacacccctgaaagtgcatggaatgagaacgaagtttttttgcatctattgtattatcctggctgtgattgttctttttgtcgtgggcttcctgacaagtgccatgagtggggagggatcttttttggcctttatcgtttcgctggtggccggggcgttcgcctctgtggtcttgcggatcgtctttatcaacctttccatgcagcgcaagttcggcaagttcaaaagacaggtatttgtcatatcgaataaggatctgttaaacagcctgtaaacactctttttatgaaaatatcggcagcgcaagcgtactgtatcttggatacggtaggatcttccatacttaccaaatccggcgaaatatccgtttcctacctgttggaattgccggaggcctattcgctggatcgctcggatctggaggaaaggcataacgagttattccgggctttccagtatgtccgaaccgggtttatacataagcaggacgtgtttttacgcaggaaattcaagccggaatatgtgatcgagggggagggctatatacagacggccgaaaggaagtatttcgacggacgggaatacctgcatcacttctgcatattgtcctttacgctttcgggactgcaaagccttgaaaaagcgtaccaggccaacccgttggcgtatagcgagaaactggcgaaggcagaccgggacaggctggccgagtttctggaatcggtggagagtgcagtttccattatccggaatatccgtggcacgcagatccggccgttgaacgtggccgaggtgaaacagcaccttttccggtacgtgaacggtttccatgacgacgagggactaagggatatacagtgttccgatatgataaggatcgggcagaagaaagggatattctttgccgtttgtgatgaaaggtatctgcccgacaggatgaaagtatatgttacggacagcactttgcaggaggcaaacagccagctttatatgtcgatgctggaaaggatcggcgttcatgtgccctgttcccatgtggtaaaccagatatggaaattcgccgggggaagctaccgggaggaactggccgagagggtgaagctgttcggccgttaccgggagtttgacaaggccataaaaagccgttatgacggcctggcctcgtatgagcaggagatcatcaacgaggaaaacgtgctttgcaaaacgcacctgaacgtgttgctgttggaggatgacgaaacgatcctgaaccgacagacggaacaggtaaaaatgattttcacgaacgccgggtttaaatattacattccaagttacgaggggctttataatatctttataggctcgatcatcggccgggaaaacaacctgaatccggactacctgtttctttcggatctgcattcgtcgctctgcatggggatcaattactctacgttccgcagcgacaaggaggggatcttgtttaacgaacggctgtatcaaacaccgattcgaaaggacgtttgggacgcagataagaaacgcattccggcacgtaacatgattatcgtcgcatcgacgggtgggggaaagtcggttacgtcgttgaacatcatccagcagaacatcgagcagaattataaacagatcgtcgtcgagttcggaaagagcttttaccagctttcgcagttgtacccggacaggtcgctgcatattgattatgacggcagcagcccgttggggatcaatccgttctatacggccgggaagcagccggacaacgagaagataaagacgcttgtaaacctggtgcttaaattctggcggtctaaggcgatcatggaggacacgaaacaagttgtttcgctgacaaagatcatttgccgttattacgaggatattcccgacggccattgtttcccggacttttacaggtacgtccaaagagaggggaaaaaactgtatgagcgtttgaatatcctgccggaatacttcgatatagacagcttcctgcacgtgtgttcggaatttatgccggggggattttatgagaacgtgtgcaagccgtccccactggagaacgatatgcagaaccgggattttatcgtctttgagctgacgaagataaagaaagatccgttcctgatttccgtcattatgacaatccttttcgatacgatagagaacaagatcctttcggatcgttccgtcagggggatgctgatctttgacgagtacgcagaaagccagtctataaaggacaccttttcaggtgcggacatacattcgacggtggcgttctgttaccagaaactccgcaaggaaaacggggcggtgggtacgatcgtccagtcattggcgcagcttcccgataacgagtacacgaaaggcattatcgccaatacgcaactcttatacgtgcttccggccaacgagatcgtttacgaccagacggtagaggcgttccatatcaagaaccggagccacgtaaacctgatgaagtcgatccgtaacgacttttcaggggtacggccgtattcggagatattcatccggtttatggatagttatgcaacggtggtacgcctggagctttcaccggaaaaattgcttgcattccagaccgacggcgagaaatggaacaaattgcaggagatttacaaggaggccggaagcatggaaacggccatagagaagtataaacaattaaaacagaaaagttatgaaacggaaatgtctatgtagtttgcttgtgatgatgtgcttttactgtacggcgttcggccagtgggtggtgtccgatccggccttgacgcagctatcgcagataacctgggcgaaagaacttaaacaggcatacgaacagtttcaagtgctgggggaatccagggacatactgactaagagcctggatctgtaccggcaggtgaacggggttatcaaaaactctaaaatggtgttgcaggtgctttccatgcagggggaaatgttggaactggcggcgaaagagtgcacccggtcggacgtgtttaccggacaggaggcatacggggaatatacgaacgtgctgaacaagatcatggaggaaagcgtgctttcctttgacctgatccggacgatcatatcgccctcggtgagcatgaccgacggggagaggataaaaattatcgtggatctggataacaagctgaaagagaaccgggacaagatgctcgacgagagagcgaggtttaatacggtcaatgacgctataaaacggatcgcagccctgaaatcaacagccaagaagtgagcctatggaagtgatcgacaatgtgattagcgcatacgaggccgtcaagatgaacggcgtcagccaggggatcatcgccctttgttcctttatcgccgttatcatcgtgatacaaaagttcgtgacggcctggaaagaggcctttagcgagggggacaagcccgtcgatatgaaacagttcttcaaactgttctatatctatatttatgtgttcgccattatcatggtcgcaccgttcgcctttacgatcgtggaaacggcacttggaaatcttcagaacgaattgataagccattatcaggaggacgtggatctaagcattgacgaggcgatcgtaacctttacaaaggactatatcgaggacgtgcaaaggcggcataactgggtagggcagcagattcaggaggttatcatgctgccgttcaatattgcggcttatacgatactcctgtacgcaacgaaatatatatttttcttctttgcgtcagcccggtatctgtatcttatactcctggagatcgtcacgccgatagccgtaatactctacatggacgagaaaacgaggcattatacgcacgcatacctgaaaaacctgtttgtctgttacatgacgatcccggcgttcctgatagccaacgccctggggtctatcattgcggagaatatcatgcacatgtgcggacagaacaaatataccatgctgggacttcttttcgctttcgtgttcaaacttttcctgtttgccaaaagcgtgaaattttgtcgtgaactgttttaaatcctgatcttatgaataaagacgatatactgaaagattttgatacactggccgaggccaagcgcaaaggagcggtaaacttaaaattgtgcctgggctttgccctggtggtggttgtcctggtgctggcctggggactggtggtgaattttacggcactggataaggtggtcgtggtggagcgatccggcgagtacctgaaaacgcacgccgaggatagcgaggctctgtttcttgccctggtgaaaaagacgtgcgccgaggcgaccgggtacgctaacagcttcgatcgcctgaacctgaaaaaaaatcaggcgcacgccgctttttatgtcaataagaacgatctgaatgccgtgttctcaaagtattataatgataaggcctattttgatgcggtgcagaacggggcggtctataaatgcgagatagacagcatccagacgatcgccggggacaatgagccgtacaaggtggctttcacttccaccctgacgatttacggcgtttcggggcagaagctaaggtttcttatcaggacgaaaggggagatcgtgagaaccacgccgcaatttccggagaacgtgacgggatttttctttaaccagtttatacagcagattgtccggatagaacaagaacctgaaaaaaagtaacactatggataaggccacgaaaacaaaaataatcctgttcgcatccctggggatcttcgtgctggtggtgctggccgttacgctggcactgaccgggaaaggggacaaccgggaagaggacgggagcgtgaacacttcgcttagggcacagcagcaaagcgattttaccctggacgatatgatgaatacggacggacggccggagtacgagaattttaccgatgaagtgtaccgggaacgttcgggagctatgtaccgggaagatccggaagtgatagccttgcaggaacagttaaggcagaaccagagagccgacagtctgaaagcggccgcagccatgaaacggagaacggcgaaagcccggccgaaaaaggaaacgccgaagaaggaagagcctgaaaggacagtaggacgtttcttttcgggggaagaaccggagaacaagggaaacacgatagaggccattgtgtcgggcgatcagaagatcacgaacgggagcgtgataaaacttgtcacgctccaagaaatgcaactgcccggcggcatggtgattaacaaggggacggccgttttcggggtggtgaagctcgcacaggacaggatcaatataacggttgaatccgtccggatcgggaacagcatctatgagatgcaaaagaccgtgtacgacagggacggactgcccggtatatatgtgcccttgaacatcaaggcggaagcgacaaaggaggcggccgacgaggtggttagcgatatgaacgttacctcttacgggacggacgttctaagtacgggggttaatgcggtgagcaatgcggcaaagagcgtgttccggaagaagaacaaccaggtcgttgtcaccgtgaagtcgaactataaactatatctgaaatgaaagctatttgttcttttatcctggcgttggccgtttcctcggccggggcgcagatccgcccggtggaaagcctgcctatcgcagtgaattattccaagacgatacatcttgtattcccctcggccgtgaagtataaccaggccgttacggacttcgtggccgtcgataacccggagagcgtgcctaatatcttgcgcataaaggccaaccggaaaagtttcagcaagcagacgaccgtcagcgtggcgaccgagggaggttttttctattcgtttaacgtgacctatgccgacagcctggagcatacgaactatttcctgcccgatatgtcgagcatacggccggacactatctatctgaatgaggtgtcgcaaacgcacctgatcgcaccggaaaaggtgatttatatagattacggcgatacctgcatacaggtaagcaaggccgagaataccgagaatatcgtgcgaatgatcgcagccacgggaaaggtggaggaatttcccaggcagacaaacgtgtcgctggcgaccgaaggggggaagttctacacgttcaacgtggattatcggcagcagccggaggcgttcgtctatgagataggggagaaacggccggagaaaaaggcgaacgtgatcctgaccgacaacattatcccggccggggaaagggatcaggtcatgagccgggtatataacgcaaaaagggggatattcaacaaggggatcgttcgcaacaagatcgttttctccgtgaacaacctgcatatatatgataacttgctcctgtttacctttgagatcgagaacaaaagcaaactgccttatgatatagattatatccggtattacatcatcgacaagaaaacggccaagctgaccgcatcccaggaagtggatcagcaggctttgttctcggagaattattcgcccaggatagaggggaacggccggatgaaatacgtgatcgctttcgataagttcaccattccggacgagaaagttttccgtatcgagatcaacgagaagaacggaggccgtcacgttctttttgacctggagaacagcgacatcgtgaatgtggaggatatttagccttgcgatccttgcgctgatccctgtcggccgggtgggggcgcaggacaaacgttcgctgatcgtggaggtgttgcagaccgcaggggattacggggacatcgccccggtgtgggggatcgtccaaaaggacggggatatttaccgtttcgtcccctctgtcgcacccctgaaagagaaataccggatctccgaccgttacggctaccggacgcatccgataagcggtgaaaggcagtttcacgcaggcctggatatggcggcggtgtatgcggctaccgtacacgccgcagccagtgggacggtgacgttcgcaggtgaaacgcccggatatgggaaaacggtagtcgttacgcaccgtttcggatttcagaccagatacgcacatctgacactcatttacacccggaaaggtgcgaaggtggaaaagggggacgtgatcgggttcgtgggaagcaccgggataagtaccgggaatcatttgcattatgaggtgataaaaaaccaaaagagaataaaccctttaaatttcatttatggaactaagtaatgaattgaaagtagaaaggatcaggctaagcctgacggcgaaaagcgttgccgaggaaatggggatcagccgccagcagctttgcaacatcgagcagtcggaaaccgcccccgtggtggtgaaatacatcgcttttctaaggagcaaaggggtggatctgaacgctctgtttgaccgtattatcgtgaacaaataaaataaatcgtatgagaaaattagttaccgccttgtgcgctttggtctttttgtcgtcttgcgacgacgaactgaaacttgtcagccgggatttttccgtgacgctgaaaagtatagacggcaatacggccgtagtggggaagcccattaattgcacccttacgatctcggatctcgatccggacaacggggatcagatattgacacgctttgaggtcagggacggggacggcgttatcctggtggataacaacgaatacagccccggagaaacgtttgaatatgatttcaaggcaaacaatcggctggatttcgattttatcccggcaaccgagggagaggcgtatatcgtcatgggggtagccagcgaactggtgacacgatccgacagcatcaaactgaaagtttccagcccggagataaacatacgtttccagaacgtgccggatcttatgctggtatcggaggaagcggaattttatttgcagcttgatacggaactttacggggtgaaagcgtcggccaggttcgtaaagggaagcggccgggtgtatatttccgggtatgatgcgacaaggggcgagggcgtggcactggaaaaaaacaacctggtgacgttccggccggatgcgaccgggcaggcggtgatcgagtttaccgtttcaagccgttacggtctgccagtgaaagagaatgtaacgatacaagtaaatcaatgaggttatgagaaaaattgccgtattatcgctctttttgtgcctggtggccgtgtgcagggcacaatatatcggaagccagtatatgaacgtgaagctgggctatatcgtggatgaaagtcaggtgcaggggacgttcggttttggaaaggtgtacaaggctttcaaggtgggggcaaaccttaattaccggaatctaaaccgggatctggtaaaggcaaacaccgtgacggtcggcccggagctggcctattacgccctgaaaggacggaaattctcgctcctggggatcgcagccgggacgatcggctaccagaaagccaaggcgaaaagcgatcttgtgtacctggagaaaagcaaggctttcgtgtatgggtacgaggtcgggataaggccggaaatgttgctaagcccgaaagtggctctttttgccgaatacaggtttgaaatgttgtttaactccattctccggaataacaaccatgtcggtctggggtgcgtgatctatttataaacttaaataattgaaggtatgagaaagtatttgtatttgtcagccgtatgcgtgtgcatggcattgtgtttcgtcggttgcagcaaggacgacgacgaaccgggaggaaaaggggcgatgtatgaggtaacgatcgagcagtcgggagatttccgcagcttcattaagtcggtcgtggtagtcgccaacggtacgcagttgaaagacggggcgacgggggaaagcctcgcaagcccggtgattctaagcgatgaagaactggccgtcgaaaaggtgacgttaagcacgacgggaaaggcgatcgagtttgccgtttccggtggtgtcgtggacggagaggacggcgttgtgaacgagccgatgcagtgggtagtgaccgtttacaaaaacggaaaggagatcgagaaaaagagccttgttttccgggacgggaaggagataagtacggatgatcttaatctgtattataattgacgtgacggcctgaatgtcccccggatgaatgttcgggggattctttttcttttgggcgttcccgtgggacggtcgggctttacgctccaagtcctcgcctaaaggctgtgggctttactctgcaatccctaacgcaattttgggtgtccgctttgtcattctgttaccgggatttgtctgcatggtgggagaaccgcaccgatcagccaggcacgcaatatcattttgtcattgccagcaggctgtttttatggtgtaacgctccgatattgctttctttttgatagatatgcaaatgcaagaagccccccggcgaaaaccccgccggaggcaattatagaaagtctgtgtatcatttccacaaaatgtggaaatgatacacagataacagttcttcaataaatatatgtgttttttgtatgcttttgattatcaggtatatatgacttgtttatttgtttggcacaatgtttgttctttgtctttcagaaaccatatgttaaatatttaaatagattggattatgaaaaaggtattatttgcagtagcacttgttatgggattgggtagttcagtggcattcgccggcaacatggtatcggatgttgagattgtggtaatggtgaacgattataagccaatcgaggtaaaggatctgccgcaggcggttcaggacacgatcaagaaggatttcgcagacctgacaatcaaggaagccgcagttgaggaaagcgaggaaggcacaaagacctacaaggtgacatttaccgatgcagaaggcacggacagcgaagtgttcttcaacgagaagggcgaagtcctgaaataaagcaaatgaacagcaaaaagagaattcaattaaactgtgtcagcaaaattaatattttattgctttgctgacacagtttgttttacagcccctccggttccggtgacttgtaaggcttgaaagcaacccgaacctccttatgctcatcgatgacgaaagtctgctccggctccacagaacgaataatgacgccattgactttccaatacatgattgtccatgatttggaatgggtagcacgaaaatgcaccgttgtgcctttcgctatttcagccggagagatcaattctcctatcttttccgcagccgtattttccaactccggtttagccgaaagctgaccttcaccctgatccacaccaaatgccagcactactttttccactgcgggaggaagaggaggatcttcatcattgtcacaagaaaccgcaaatagcgtaagcccatacaacaaaacaacgacaaaagagagataaccgatctgtctcattaagccgatttcttttttttccatagcagttttcataagcttattttttgtttacgaccgcataaagataacaatttgttggctaaaagggtttattaaatatagtgtttatcacttaatgatttaaggttaatatcctgtaggttgcagggagaacaaacgggaacagccgttcccgcccctttcggattgtatgccagcacccctaaaaaatgccttgtcaaagacaagtagtaacacgcctgtgttacgttttcgacttttcagccgtccgaccgccaccccgtccgatttgacttgcctttgaggttatatatgcaaatgcaggaagcccccagcgaaaaccccgccgggggcaatatttttaaacaggctttaaaaatacacgttcagcataaggtataaaaagacgttcttatccttagtgctgtggtgttcaagttcgaattggattttgtcctcacgtgcgagccaacctattgcgctggcagcttccaggtcattgaaccctgttgcatccttgacttcttgtaaagtccattttcggtgtacgtctgtgcttaataagcgccagagtttccctgcgttctctccgatgatgtgcttatccatactttgttatttgttataaattgataaatggtttcaaatggataatgaaaagcacttttacatcaatagtgttacgcactgcaaacattccagcgtttatcgtttatccttttgatacgttgtatcactctttcctgatatacagcaaaccgtttcgccgttcgatgtatatcttattctcacgtgcgagccacccggcggcgagtgcggtttcatcagccgcaaaatttagccttctcgcaagctcggcaatggttataagtccggtttctcctaatttttcaaagatgatcccggctttaaatcctatatttgctttttccatgataaagtttatattttaatggttcaaaaatatgtatatattttattattcaaagcgtgtagaaatacaaattagaatttaccccgttaataaattttaacaatatagatattacgttctattcatttcattttgcggatgaaaagaactttacaactctatcttatagggggttaatttagtaataccttactgaaaaacaacgctctgaaacattccctgtcaccgttaaacaggcacaaatatctcccctttacatcctcaaagaagatataatacaggcaggaattaatgccgaatctatttgttccgatgatctttgcatggcttaatttttggcgtacttgtttttccgtcagcccttcgatgtatatcaaccctttcatgtttcccatttgtccgggggtgatcttaagcgaactaagtagttctttgaggtcaaacagcttaggagtgtttgacaagcagtttagggactgttttcgctgtgtcttttccattgctataaggatttcgcttgtgccctacaccaagccaataaacataaaaaaagcggaagggcattcgcctatccgcaagcgagggagtccaagccctatacacacaaacaggagataccccccgcatatcacggggagcatttatctgtttctctgtgtataatgcaatttttggacttttcgcttgcacagaaatcagctaaacgctttttttctcaaaatgtaaaaacacggtcggtttacgaccgcacaaagataacaaattgttggctaaaagggtttacgaaccttttatttttttgccctattatcttaaattcagtgtttatgaaataccaagttctttttgaacatccttgtttgcagatgaaaagatacttttgcagtaccttaatgttgtccgcatggtgggagaaccgtaccgatcagccgggtgcgcaatatcattttataataaccgataggttgtttttatggtgtaacgccccgatatcgctttctttttttgtatctttatgtagaaaatattgcgtattaactgttaatttcgaaaaatgatgtatctttgcggctttcaaaattgtaagtatagaatttataattaaaatattaggtttcaaaagtataaaggtttttggttagttgctctttctggggtagcagatattttatagtaagttatatgcaaaaaaaacagaaaaaagttggaaatattgattttttttataaacacacacacacacacacacacacacacacacgagtgtgtgtagaaacctatatttaagacttttcaaggtaattctcctttcttttttatcgtgttccgttcttttcgtttccgctcaagagacacgggattccgtgaaaatatatttccacaagggtagaacggaaatagatctctctttacgggataacaggcggtcgttagagaagatcagccgccggatattggaatacaggccggacacggctttctctatctgcaagattatggtggtcggtgcggcgtcccccgaggggagcatcgaactgaacaggcgtttgtctgaactaagggcgaaacgtttgttcgatcacgtgtgcggttactcgccgctacccgacacgctgaaaacgtcggtattcgtggggcgtgactggaggggattgctgcgtttggtggagaatgatcccggaatgcccggtaaatcggaaacggtggagcttctgcaaggtattgtttccgaggtggaagcaagtggaaacggggagaacaagataaatgaattgaaacgcttgcggtacggcatcccatatcggtacatgtatcggaatttgtttccggatcttcgtgcatcttgtttgtatatttggtatgaatcgaaacggaagtcgttccctgccatgcaggttatctcccacctgcatgatacattgtacttaccttgtaagctttctccgttcggtggagaaacgggcggtttccagtctggggaaaaacatgggcctttttacatggccgtgaaaacgaacatgctttatgatgcgttgcttgtgccgaatataggggtggagttttacgtcggaaagaactggtccttggccgggaattggatgtatgcctggtggaaaagcgaccggcaccataactactggcggctctatggcggggatctggaagtgcgccgctggtttggcaggaaagcgtcggaaaaaccgcttgcagggcatcacatcggtctctacggtcggatattcacctacgacttcgagaccggggggacgggctacatgggcggcaagcctggcggtacgctttgggataagatgaactattcggtaggtctggagtatggttattcgctgcccgtcgcccgtcgtctgaatctggatttcgtcatcggtgtgggctattggggtggagaataccataaatatgcccctatcgacggccattatgtctggaaggagacccgacggcgccactggtttggtctcacggaggcggagatttcgctcgtatggctcttgggccgtggcaactacaacgagaagaaaggaggcaggcagtgatactgatacgctttagtctggtgttcatatttttatggatgggactgacggcttgtgagcataaggatttatgttacgatcatccgcactttgccacggtgcgggtagtattcgactggaccaagatttccaatcatgacaagcctgaaggcatgagggtcgtattttatccgaccgatgatgaaagcaacacgtggatatttgatttccccggaggagaggatggggaagtggagctccccgagaatgattaccgggtaatttgcttcaactacgataccgacgggatggtttggaaagaaaacggcagttacacgctttttactgccgatacgcgtgatgttcggtcgccggataaccagacaatggccgtcaccccaccctggctgtgtggcgaccatatagacagagttatcctcaaggacattccggaaggaagcacgaagataatacggctaactcccgtaaacatggtatgtcactacacgtatgaggtgaacggaattcggggactggacagggtggcggatttgcgggccgctctttccggcatgtccggctcgcttaacatgtccggcgacagtttgcccgccgatctttcggagagcctgctctttgatggaatggtttcacggaatcagattataggcggattctatacgtttggacattccgcacttgaaggagagcccaatgttttccggctgtatctcaagaaccgttcaggcagcatgtctgtattggaacaggacgtgagcgaccaagtgcatgatgtcccggtagcggggcatatcggtgatgtgcatctggtgttgaattttgattatgaggtaccatccgagccgggaagtggcggtccgggatttgatgtggacgttgatgattgggatgatgtgaatgtggatatagtgttgtaatcgagttttattaattatatttattaacaattttattcattttatgaaaaaaagtacagtaatgctttgggcaatctttggagtactactgatgagttgctcagaagaggagattgcgaatgtggaaacctcctcacgaaatgcgattggttttaacgtgttaagtaatgcggcggaaacgagggctacccctactaccaacaccaacttgaagaacaccgatttcgacgtgttcgcttttacggcggatggtactgccttcatgggaaagaacgacacggaatttgaacatgacggagtgaagatcgtgtacaaaaatggtaagtgggattatgacaatgcgagcgaccttcgttattggcctaccgaggctttggacttttacgcgttcaatcccggaacggttagtgaagacatgatggcgttttatagttgggaagccaccaaggatgtccaaaagataagttatacctgcatggatgagtatggaagcggtactactcatgcaaactacgacgtgatgtatgccatggccaaaggccagacaaaagacatgaacaacggaatagtgaaatttaatttcaaacatattctgtcacaggtcgtattcaaagcaaagacccaatacgataacatgcaggtggacatcgacgtgatcaagattcataatttcaaatttgccggcgccttcacattgcctgcggcagccgatggaacgggaagttggagttcgtctgatttagcatttccacacgcatttaccgtagtgaaaaacgcaaacatcacggtcaatagcaataccgaagctaccgatattaccacaaatactcctatgctgaatataccacaggagctgactgcttggaaagtttccgaaacagccaccaaaagcaagttggaggccgataatgcgaagcaatgttatctggagatcgcctgcaaaatccggcagtccggagcttatctgttgggttcagcaagcgaatacaaaacaatttatgttccgttcggagatacatgggaacagggcaagcgtcatatctacacgttgattttcggtggtggttatgatgatcagggagaggctgtgttgaatccgatccagtttgatgcagagacaacaggctgggttgatgcagataaagacgtaaacgtccaaccttaatccaaacgtttattaatatcgtacccgttggcaggttaaacatacgcaagattaaccctgccaacgggttttttatagggaatcgcattttcaatatactttttgatgtgtaggggtgctttttgcgtacctttttcctggagaatttcttttgtggagtgtttgccctgtatattcccgatacgctgttatagcagggaactgtttttatccaagtagtcagatatagcctcttgcaataccgattttagcgaatcgcctttgcggatcgcgatcagctttaagttctggtgataggtttcgtccatgacaaaattacaatgcacggttttcgctttcttcggcttttgagcctctttttgcccggccgtggattggatcaggctgtctaatcccccggtcagcccggccgacatactgttttttaaatcgttcttctttgccatatggttactttatttctgttagcagttcgttacatacaccctcgtagtcctctgccccggcagatttgggggcatagtgaaaaatatcctgcccttgtgtcggggcttcggccagcgcaatggagttgcgtacatgggtgctgaacactttgccctggaatgtttcctgtaccagttccgaaacgctcttgttcaggttctttcttccgtcgtactgggttatcagaaccccggcgatcgaaagatccgagttcaaacgttgctgcaccttgtggactacctgcataagtttggccattccacgcattgccaggaattgagcctgaaccgggataatcaaccgatccgaggccgtcagtgcgttaagcgtgagcagcgacagcgagggaggacagtcgatcaggatataatcgaatttctccttttggcctttgataaggtgggctaagataagctcccgtccggcctcgttgattaactccgtttcaacggccgagagatccaggcaggagggaacgacgctaaggccgtccttatgctcgtagatcggcaggtcgtattcgcctttcatcgcaccgtagatcgtttggggaagctccgcagagaagcccagcgattcggtcaggttggcctgcccgtcaaggtcgatcaaaagaaccttgtaccctttttgacgtaacgcaccgcccaggttgatcgtggttgtggtctttcctactccccccttgtgattcaatacggagattacttttgctttgctcataaattcagttttttaaatggttgtcatgtccgcaaatatagttaaaatactaatatactaaaatactaaaagtagtatttttcgttttttgcgtgttttttgtgccgatcggggaagcgtaccgaaataaatcctgtttccgtctttcggtacgttttgtttctgtaccggaattaatcaaagattagaataaccataaatgtttttatacgttaatcatacgtatgtatatatgtatatatttgtttttgagtatctttgtgtgctataaataaaaaataaaagacatgagcacttattcgtataaaaatccgaagtttataaattcccctaaaggggtggttgaagttgtagaagtgatttatgacggcaaagatgatccggcctattcactggctattattaaatgggaaaatacctataaattaggtattcgttggaacatcgcttatagtgaatgggacgattaccgcaaacagaacgggcaagatgaatgtataggtaatccacagtcgagaggtattcccacctggtttgttttgcccgatgatatgatgtttggtgagaagttcagcggtgcgatgcagaggcttgatgaattaagaaaaggtaagtaagcaatggaacagggagaaatcatattataccagccggatgaagcggtcaagttggaagtgaggttggaagatgaaaccgtttggctgacacaagaacaaatagccgacttgttcgggactaaaaggccagctattaccaaacatttgaacaacatttacaaaagcggcgaacttgatatagatagcacatgttccattttggaacatatgggtaatgacggcaaacagagatatactactaaatattataatcttgatgcgattctgtcaataggatatcgggttaatagcaagaacgcaaccctttttaggaaatgggcgaatagtgtattaaaggattatcttttaaaggggtattccattaataaacgtttgtcagaacttgaaaggactgtcgctcaacataccgagaagatagattttttcgtgcgtactgccttgcccccggtcgaaggcattttttataacggccagattttcgatgcgtacaagttcgccaccgatctggtaaagtcggccaggcgttcgatcgtgcttatcgacaactatgtggacgaaacggtcttgctcatgttgagtaaacgcagcgtcggtgtctcggccaccatttacacacagcgtatcacgcagcaactccaacttgatcttgacaggcataacagccagtacccacctatcgacatcaggacgtaccgggacagtcacgaccgtttcctgatcgttgatgaaacggacgtgtaccatatcggagcctcgttaaaggatctggggaaaaagatgttcgctttctcaaagctggatattccggcggctgtgataactgacctgttataatatcccttgtcttatgaatgtggttatctattcacgtgtaagctcgcagtcggccaggcaatcgaccgaacggcaggttgtcgatctggagaggttcgcagccggacgggggtacgaggtgacggcggtctttgaggaaaagataagcggacgaaaggccaatatcgaacgcccggttttaagccgttgcctggaatactgcacagatccgcagaaccgggtggatatgctgttgctgaccgagatctcacgcctggggcgttccaccctggagatactaaaggcactcgatacgctgcacacgcataaaatatgcgtgtatatccaaaacttgaacctggaaacattacggccggacaagacggtaaaccccttgtcctctctgattactaccctattgggggaactggccgcaatcgaacgccaggggatcattgaccgccttaatagcggacgggagttgtacatccagaaagggggcaggctgggacggaagccgggaagccggaaaacggccgaacagaggaaagaggaataccgggaggcgatcgccttgttgaagaaaggctactcgatccggaacgtggcgaagctcacgggaaaggccgtttcgacgatacaacaagtaaaaaaagacttcattaatagctgattatcggaacaaatattgtatttttgcaacaaatacgagtaaatgcacgtaattaaaaacgagtatttgcacgaaaataatcagaatctaaataaaaacggttatggcaacaatggcggagaaaatggcggcttcactggaggaattaagaaagctccaggagaaagaccgctgtgtcgtcctgcaagggacggccgagatagggaggacgcatttaacccggctgttggataacggctggttgcaggaagtaatgaaaggctggtatattgcggccaggccgggaacagagggggacacgacggtctggtacacctcattctggtattttattgcgaaatacgcggccgtccggctgggggaacaatggtgcttgacggccgaccagtcgctggatctgtattcggggaaaacgaccgtgcccgtacaggtggttatcaagtcgccgaaaggccataacaacacgcaaaagctgatgtatgatacctcgcttttggtgtttcaaagcgagattccggatcaggtatataaagagccggaatacggccttaatctctatccgcttgccgaagccctggtatataccacgccgagatatttccaggtggagaagatcgcagcccgcacctgcctggccatgatacgggacgctgccgatatattaaaggtactgacgaaaaacggggcttcgcttcgtgccggaagaatagccggggcgttccggaacatcgggaacagcgaaatcgccgacagcatcgtttccacgatgcgggggttcgggtatgacgtgagagaagaagatccgttcgaggatcagccacgaacgccccttgtttatgaggtgtcgccttacgtcacacgcttgcgcctgatgtgggagaacatgagggataaggttgtagaactgtttcccgaagcccctggaaagattgatgacgtggaggggtatttgagatccgtcgatgaaaagtattcggaggacgcttatcattctctctcgatcgagggatatagggtttccccggaactgatcgagaaagtgcgtgtcgggaactggaagccggaggaagaagataaggagcataaaaacgcactggtggcacgtgggtattaccaggcgttccaggctgtcagggggacgatcgccgatatactgaaaggaaagaacgcaggggaagcggtaagggcggatcatccggtctggtatatgcagatgtggatgccgttcgttacggtggggatcttgcaaagggaggatcttgtgggttatcgtaccgggcaggtctatatacggggatcgcaacatatcccgttgaacccgaaagcggtcagggacgctatgcccgtccttttcgatctcttgaagaatgagccgcacccggcggtaagggctgtcttgggacacttctttttcgtgtatattcacccctatatggacgggaacgggagaatgggacggttcgtcttgaacgccatgcttgcgtctggaggctataactggacggttgttccggtggagcgtagaaaagaatatatgaaagcgttggaaaaggccagcgtggagggggatatttcggagtttacgaaagtaatcgcatcattggtcaagtaatgatatttataataatgacaatatgaaatcaacggaatatatcgaatgggataaactggagcagatcccgttctgcctttgccggatcgcggaggatgaagaaaaccaggaaatagatgtttattatctggataaacgggtttgccatgattacgatcatgtggggcattattttcgtactgcgataatcatgtttagaaggatcaggaacataacggccgattgggtgaacctcaaaaacctttggttgctaagggactgtatcagggagaattttaatcatggcctggaggtggacgatctgatcttcggggaaacattcgacggtgaggatccggaaactataaagccgctaaccaaagaacggctgttcaagattaaaaaggtgattcaggaaaaagatccatacgctaccgtatagggggggagtatataaaaacaaaaccctggagtttgtcttgcgacagggacaacaggggattactacgcaaatatagggagtatatctcgtttatacaaggattttatggcttttctggattgattttaaaaagaatagaggcttgttccctggaatgtagtatttactacttttagtattttagtatattagtacgcttttcttttggaggatttgggcgttcccgtaaacggtcgggctttccgcctccaagtcctcgcctaaaggctgtgggctttccactgcaatccctaacgcatgaatacacccactcacccgattaaggtaggctatttccggatagagccgttcggtgaagccagccggataaaagccgcctgacttttcactgatcggcacaatccgggtactccggtagccgattgtcgaacccggttttaaaccccctttcaggggagagaggtattttagtttttccggcgtggccgccttgcgtttaccttgaccggggtttttgtctgcaaaggtcaaacgtttttttccatatcccaagttgagccgcaagcggcttttagaaaaaatctccagcctggctaaagccaggtagtatttttcctaaaaaacttgtgctatggaaaaaaacgtttggggtgttctcatacaaaaacccctcggaactcaaggtaaacgcaagatgaccacaccgacagccccgaaaaaccaaaatgaggttgtcttgatacaaaaacatcggaatcttaggcatccgcaaggttttttgggggggagagggtgcgaggtggggagtttactatcggaaaaaatcaggtagtaaatactaaatttagtatatactaaaatactactttttcatataattatctgataaatagttagttaaatgttgtttaaatacttgcatatctcaaataaaagtgctatattagaggtatagaaaaagagataacaacctttagaactggctcacccaccaggataaagaacgggggacgtagttatgaaaactgattatttcgacaaggcagcgcaacagtttgcaaatttgatgatcgagaagatccagcaggtagaggacaattggcaaaagccctggatcaccattgcggcgaatacacgcaatttctttccgcagaacctcacaggacgcaggtacaccggaggcaacgcctttttactccttttcctttgcgagaaattccagtaccagacacccgtatttatgacattcaaccaggcgaaagaggctgggatctccgtgctgaaaggatcgaaatcgtttccggtctattacttccttttctacgtgtaccacaaggaaacacgcaagaaaataacctttgaggaatacaaggcgttaagccgggagcagcaacaggaatacaacgttataccgacctataaatattactcggtctttaacctggatcagaccaatttttcggacgtgaggccggaagaatgggaagcgttaagggagaagttcagagggggacaggcagagcagcccgacgggacaaccggagaggcgcaccccgaaatagatgccatgctggaggcgaaaagctggttttgcccgatacgggagcagcaaggcgacagggctttctattctcctttagccgattatatcgtagtgccgcttcgctcgcagttcgtcgatatgcagagcttttacgaaacgcttctgcatgaaatgggacattccacgggacaccccacccgattgaaccgggatcttgcgcacccgttcggatcagaggaatacggcaaggaggaattaacggccgagttcgccgcagccctggcaggaatgtttttcgggatagccgaacatatacgcactgaaaacgccgcttatctgaaaagctggataaaggcgatccgggaagaacccaagttcattttaaacgtactggccgacgctatgaaaattgtcaagatgatagccgggaaactggatatctccgtagaaagcgaggaaacggccgcagatgaagcggaacaggtggcatgatacaaggaagatcccccgaaagggggacagcctttcggggtgagttctttttggcactcgccccaaaaagaaccaaaaaacgctttgccgctacactccgtttcgctttcatagctctgataatgcgttttttgattggatagatatatatttgtagataaaataatgtaat

(19) United States
(12) Patent Application Publication
(10) Pub. No.: US 2023/0302062 A1
(43) Pub. Date: Sep. 28, 2023

(54) COMPOSITIONS COMPRISING BACTERIAL STRAINS

(71) Applicant: 4D PHARMA RESEARCH LIMITED, ABERDEEN (GB)

(72) Inventors: IMKE ELISABETH MULDER, ABERDEEN (GB); ALEXANDER STEVENSON, ABERDEEN (GB); IAN JEFFERY, CORK (IE)

(21) Appl. No.: 18/295,874

(22) Filed: Apr. 5, 2023

Related U.S. Application Data
(63) Continuation of application No. 17/226,148, filed on Apr. 9, 2021, now abandoned.
Application No. 17/226,148 is a continuation of application No. PCT/EP2019/077332, filed on Oct. 9, 2019.

(30) Foreign Application Priority Data
Oct. 9, 2018 (EP) 18199455.9

(51) Int. Cl. A61K 35/741; A61P 37/00; A61P 1/00; A61K 9/19; A61K 9/48

(52) U.S. Cl. CPC A61K 35/741; A61P 37/00; A61P 1/00; A61K 9/19; A61K 9/48; A61K 2035/115

(57) ABSTRACT

Provided are compositions comprising a bacterial strain of the genus Bacteroides, for use in a method of increasing the microbiota diversity and/or inducing stability of the microbiota of a subject.

### CROSS-REFERENCE

This application is a continuation of U.S. application Ser. No. 17/226,148, filed Apr. 9, 2021, which is a continuation of International Application No. PCT/EP2019/077332, filed Oct. 9, 2019, which claims the benefit of European Application No. 18199455.9, filed Oct. 9, 2018, all of which are hereby incorporated by reference in their entirety.

### SEQUENCE LISTING

The instant application contains a Sequence Listing which has been submitted electronically in XML format and is hereby incorporated by reference in its entirety. Said XML copy, created on Mar. 30, 2023, is named 56708-749.303_SL.xml and is 65,452 bytes in size.

### TECHNICAL FIELD

This invention is in the field of compositions comprising bacterial strains isolated from the mammalian digestive tract and the use of such compositions in the treatment of disease.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 1500 different phylotypes dominated in abundance levels by two major bacterial divisions (phyla), the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host and additional health benefits. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD subjects whilst numbers of E. coli are increased, suggesting a shift in the balance of symbionts and pathobionts within the gut [6-9, 16].

In recognition of the potential positive effect that certain bacterial strains may have on the animal gut, various strains have been proposed for use in the treatment of various diseases (see, for example, [10-13]). A number of strains, including mostly Lactobacillus and Bifidobacterium strains, have been proposed for use in treating various bowel disorders (see [14] for a review and see [15]).

The relationship between different bacterial strains and different diseases, and the precise effects of particular bacterial strains on the gut and at a systemic level and on any particular types of diseases, are poorly characterised and results to date are variable and pose more questions than provide answers [16].

While the term ‘dysbiosis’ has been used in the literature to generically define deleterious fluctuations in the microbiome, there is no universal definition of what does or does not constitute ‘dysbiosis’. A more accurate and verifiable metric to assess perturbations in the microbiome is ‘microbiota diversity’ Loss of diversity is also measured by reductions in the Shannon Diversity Index. As those skilled in the art will be aware, the Shannon Diversity Index accounts for both the abundance (i.e. changes in the the populations of different OTUs present) and evenness (i.e. how numerically similar the populations of different OTUs present in the microbiome are) of species present in the microbiome. A significant variation in either abundance or evenness from the ‘healthy’ or ‘normal’ microbiome in a population equates to dysbiosis.

Reduced microbiota diversity is reported in recent studies of obesity, inflammatory bowel disease (IBD), irritable bowel syndrome (IBS), type 2 diabetes and frailer older people [20]. In particular, references [17] and [18] teach that a reduced microbiota diversity is strongly associated with IBD and reference [17] furthers summarises studies concluding that increasing the microbiota diversity has curative effects on IBDs.

Re-establishing the healthy microbiota can be difficult, however, as the bacteria in the gut are resistant to colonisation. This poses a challenge when trying to treat the microbiota of unhealthy subjects by increasing the diversity of the microbiota [19]. The accompanying loss of microbial metabolic function is assumed to be a contributory factor to the symptoms of these pathophysiologies. In contrast to healthy adults in whom the microbiota is stable, the microbiota of unhealthy subjects such as those suffering from IBD, IBS and frail elderly subjects is unstable [16, 20].

There is a requirement for the profile effects of gut bacteria to be positively modified to permit the treatment of diseases or conditions characterised by reduced microbiota diversity and/or evenness.

### SUMMARY OF THE INVENTION

The inventors have developed new therapies for treating and preventing diseases and disorders by increasing or maintaining the intestinal microbiota diversity in a subject. In particular, the inventors have unexpectedly identified that bacterial strains from the genus Bacteroides can be effective in increasing or maintaining the diversity and/or evenness of different types of bacteria in the distal gut of a subject.

As described in the examples, an IBD patient population treated with an organism from the species Bacteroides thetaiotaomicron experienced a statistically significant increase in their microbiome diversity and evenness. Additionally, the examples show that treatment with compositions comprising Bacteroides thetaiotaomicron increased the stability of the microbiota in IBD subjects throughout the course of the study.

Therefore, in a first embodiment, the invention provides a composition comprising a bacterial strain of the species Bacteroides thetaiotaomicron, for use in a method of increasing or maintaining the microbiota diversity. Similarly, there is also provided a method of increasing or maintaining the microbiota diversity in a subject comprising use of a bacterial strain of the species Bacteroides thetaiotaomicron. Preferably, the subject has reduced microbiota diversity and/or stability.

The term “increasing or maintaining the microbiota diversity” is used herein to mean increasing or maintaining the number of different types of bacteria and/or the evenness of the different types of bacteria in the microbiota of a subject. In some embodiments, the microbiota diversity is increased. In some embodiments, the number of different genera of bacteria in the microbiota is increased. In some embodiments, the number of different species of bacteria in the microbiota is increased. In some embodiments, the number of different strains of bacteria in the microbiota is increased. In some embodiments, the microbiota diversity is maintained. In some embodiments, the number of different genera of bacteria in the microbiota is maintained. In some embodiments, the number of different species of bacteria in the microbiota is maintained. In some embodiments, the number of different strains of bacteria in the microbiota is maintained. In some embodiments, the number of genera, species and strains in the microbiota is increased or maintained.

The increase in microbiotia diversity may be for non-acetogenic bacteria. It may also be for both acetogenic and non-acetogenic bacteria. Such bacteria are well known in the art. Briefly, acetogenic bacteria produce acetate as an end product of anaerobic respiration or fermentation.

In some embodiments, loss, increase or maintenance of microbiota diversity may be quantified by a measurable reduction, increase or maintenance, respectively, in the number of the sequence-based bacterial classifications or Operational Taxonomic Units (OTUs) in a sample, typically determined by 16S rRNA amplicon sequencing methods. In some embodiments, loss of diversity may be measured by reductions in the Shannon Diversity Index. Conversely, in some embodiments, an increase of diversity may be measured by an increase in the Shannon Diversity Index. Similarly, in some embodiments, maintenance of diversity may be measured by the same result in the Shannon Diversity Index.

In some embodiments, the evenness of the different types of bacteria is increased. In some embodiments, the relative abundance of the different types of bacteria in the microbiota becomes more even following administration of a composition of the invention.

The inventors have also developed new therapies for treating and preventing diseases and disorders by inducing stability of the intestinal microbiota. In particular, the inventors have identified that bacterial strains from the genus Bacteroides induce stability of the intestinal microbiota. By “induce stability” is meant that the microbiota diversity remains stable and also the relative numbers of the different genera in the microbiota remains stable. Thus, the relative numbers may fluctuate by less than 10%, less than 8%, less than 7%, less than 6%, less than 5%, less than 4%, less than 3%, less than 2% or less than 1%.

Stability of the intestinal microbiota is important as a number of diseases and disorders, including IBS and IBD, are characterised by reduced stability of the microbiota. As described in the examples, oral administration of compositions comprising Bacteroides thetaiotaomicron induces stability of the microbiota in stool. Therefore, in a further embodiment, the invention provides a composition comprising a bacterial strain of the species Bacteroides thetaiotaomicron, for use in a method of inducing stability of the microbiota in a subject. Similarly, there is also provided a method of inducing stability of the microbiota in a subject comprising use of a bacterial strain of the species Bacteroides thetaiotaomicron.

In some embodiments, the relative numbers of the different bacterial species in the microbiota of a subject becomes more stable following treatment or prevention with a composition of the invention, for example in a subject diagnosed with a disease or disorder characterised by a reduction in the diversity of microbiota. In some embodiments, the relative numbers of the different bacterial genera in the microbiota of a subject becomes more stable following treatment or prevention with a composition of the invention, for example in a subject diagnosed with a disease or disorder characterised by a reduction in the diversity of microbiota. The stability of a subject's microbiota can be assessed by comparing the microbiome from the subject at two different time points. If there is a difference in the microbiome, this can be indicative of disease or of a disorder being present. In some embodiments, the two different time points are at least three days apart (e.g. at least 1 week, 2 weeks, 1 month, 3 months, 6 months, 1 year, 2 years apart). In some embodiments, the two different time points are 3-7 days apart, 1-2 weeks apart, 2-4 weeks apart, 4-8 weeks apart, 8-24 weeks apart, 24-40 weeks apart, 40-52 weeks apart or more than 52 weeks apart. In some embodiments, more than two different time points are used, e.g. three, four, five or more than five time points. Suitable intervals are chosen between the various time points, for example, as set out above.

The bacterial strain may be Bacteroides thetaiotaomicron and is preferably the strain deposited under accession number NCIMB 42341. This strain was deposited with the international depositary authority NCIMB, Ltd. (Ferguson Building, Aberdeen, AB21 9YA, Scotland) on 3 Dec. 2014.

Further Bacteroides thetaiotaomicron strains for use in the invention is the type strain ATCC 29148. The 16S rRNA gene sequences for these strains are disclosed as SEQ ID NOs 2. A further preferred Bacteroides thetaiotaomicron strain for use in the invention is the strain described in EP1448995. The accession number for the 16S rRNA gene sequence of Bacteroides thetaiotaomicron strain WAL 2926 is M58763 (disclosed herein as SEQ ID NO:3). Other suitable Bacteroides thetaiotaomicron strains have the 16S rRNA sequences of SEQ ID NOs 4-12.

In some embodiments, the microbiota diversity, evenness and/or the stability of the microbiota refers to the microbiota diversity, evenness and/or the stability in a stool sample from the subject. In some embodiments, the microbiota diversity, evenness and/or the stability of the microbiota refers to the microbiota diversity and/or the stability in the distal gut of the subject. In some embodiments, the microbiota diversity, evenness and/or the stability of the microbiota refers to the microbiota diversity, evenness and/or the stability in the gastrointestinal tract of the subject. In some embodiments, the microbiota diversity, evenness and/or the stability of the microbiota refers to the microbiota diversity, evenness and/or the stability in the caecum. In some embodiments, the microbiota diversity, evenness and/or the stability of the microbiota refers to the microbiota diversity, evenness and/or the stability in the colon.

In some embodiments, the invention provides a composition comprising a bacterial strain of the species Bacteroides thetaiotaomicron, for use in a method of treating or preventing a disease or disorder associated with a level of microbiota diversity that is reduced relative to the microbiota diversity of a healthy subject, or a population of healthy subjects. Such diseases are well known in the art and include, for example, IBS, IBD (such as Crohn's disease and ulcerative colitis) [21], cancer (for example colorectal cancer, or other cancers for example where a reduction in microbiota diversity is observed with concomitant cancer therapy treatment including chemotherapy), obesity [22], autism, allergy, celiac disease, infectious diseases, and graft versus host disease amongst others [23]. The invention is useful for treating these diseases. Preferably, the compositions of the invention are for use in treating IBD, in particular Crohn's disease, or cancer. Whilst these conditions may be associated with reduced microbiota diversity and/or stability this is not an inherent feature of these diseases as patients can suffer from these even if their microbiome diversity/stability is unaffected. A skilled person can easily ascertain whether a patient suffering from any of these conditions has reduced microbiota diversity and/or stability relative to the levels in a healthy individual, or a population of healthy individuals as explained in further detail below. Thus, in embodiments of the invention, the subject to be treated, who may be diagnosed with one or more of the diseases discussed therein has reduced microbiota diversity and/or stability.

In some embodiments, the treatment or prevention using a composition of the invention results in the microbiota diversity, evenness and/or stability increasing to the levels corresponding to or greater than those present in a healthy individual, or a population of healthy individuals. A healthy individual in this context may be someone who does not suffer from a disease which is associated with reductions in microbiome diversity. A healthy individual may be the subject being treated prior to the onset or diagnosis of their disease; administration of the compositions of the invention may cause the diversity, evenness or stability of their microbiome to revert to their former, pre-disease levels.

In some embodiments, treatment or prevention using a composition of the invention results in the microbiota diversity, evenness and/or stability increasing to levels corresponding to or greater than those present in a population of healthy individuals.

In embodiments of the invention in which changes in microbiome diversity are determined with reference to a healthy individual or a population of healthy individuals, the healthy individual/s is/are resident in the same geographical region (e.g. resides within a 200 km radius, within a 100 km radius, or within a 50 km radius) as the subject, is of a similar/same age to the subject and/or is of a similar/same race to the subject. Similarly, the invention also provides a method of treatment or prevention of a disease or disorder associated with a level of microbiota diversity that is reduced relative to the microbiota diversity of a healthy individual or population of healthy individuals wherein the method comprises administering a composition comprising a bacterial strain of the genus Bacteroides.

The levels of microbiota diversity in a healthy individual are well known in the art and can be determined by a skilled person using methods known in the art (see, for example, reference [24]).

In some embodiments, the subject is an infant or child with a reduced microbiota diversity compared to a healthy infant or child (or population thereof), respectively. It has been observed that some children who develop a disease associated with a reduced microbiota diversity later in life have a reduced diversity of faecal microbiota as 1 week old infants [25]. Thus, in some embodiments, the infant is less than 1 week old, is less than 2 weeks old, is less than one month old, is less than two months old or is less than four months old. In some embodiments, the subject is an infant who has not been delivered via a vaginal birth. For example, in some embodiments, the subject is an infant who has been delivered by Caesarean section. Reduced microbiota diversity has also been reported in frail elderly subjects. In some embodiments, therefore, the subject is an elderly subject, for example, a frail elderly subject. In some embodiments, the subject is 65 or more years in age (e.g. 70 or more, 75 or more, 80 or more, 85 or more or 90 or more years in age) [20]. The subject may also be an adolescent. For example, the subject may be between 10 and 19 years of age.

It has been estimated that a healthy human individual has approximately 101 different bacterial species and 195 different bacterial strains in its microbiota [26]. Accordingly, in some embodiments, the composition is for use in treating a subject having fewer than 101 different bacterial species (e.g. fewer than 100, 99, 98, 97, 96, 95, 94, 93, 92, 91, 90, 85, 80, 75 or 70 bacterial species) and/or fewer than 195 different strains (e.g. less than 194, 193, 192, 191, 190, 189, 188, 187, 186, 185, 183, 180, 175, 170, 165, 160, 150, 140 bacterial strains) in its microbiota. In some embodiments, the treatment or prevention results in the microbiota diversity increasing to more than 80 bacterial species (e.g. more than 85, 90, 91, 92, 93, 94, 95, 96, 97, 98, 99 or 100 bacterial species) or to 101 bacterial species. For example, in some embodiments, the treatment or prevention results in the microbiota diversity increasing to more than 90 bacterial species. For example, in some embodiments, the treatment or prevention results in the microbiota diversity increasing to more than 95 bacterial species. For example, in some embodiments, the treatment or prevention results in the microbiota diversity increasing to more than 97 bacterial species. For example, in some embodiments, the treatment or prevention results in the microbiota diversity increasing to more than 99 bacterial species. In some embodiments, the treatment or prevention results in the microbiota diversity increasing to more than 160 bacterial strains (e.g. more than 165, 170, 185, 186, 187, 188, 189, 190, 191, 192, 193 or 194 bacterial species) or to 195 bacterial strains. For example, in some embodiments, the treatment or prevention results in the microbiota diversity increasing to more than 175 bacterial strains. For example, in some embodiments, the treatment or prevention results in the microbiota diversity increasing to more than 185 bacterial strains. For example, in some embodiments, the treatment or prevention results in the microbiota diversity increasing to more than 190 bacterial strains.

In some embodiments, the treatment or prevention results in the microbiota diversity increasing by at least one bacterial genus (e.g. by at least two, three, four, five, six, seven, eight, nine or ten bacterial genera). In some embodiments, the treatment or prevention results in the microbiota diversity increasing by at least one bacterial species (e.g. by at least two, three, four, five, six, seven, eight, nine, ten, 12, 15, 17 or 20 bacterial species). In some embodiments, the treatment or prevention results in the microbiota diversity increasing by at least one bacterial strain (e.g. by at least two, three, four, five, six, seven, eight, nine, ten, 12, 15, 17, 20 or 25 bacterial strains).

In some embodiments, the invention provides a composition comprising a bacterial strain of the species Bacteroides thetaiotaomicron, for use in a method of treating or preventing a disease or disorder associated with reduced stability of the microbiota compared to the stability of the microbiota in a healthy subject (or compared to a population of healthy subjects). By “reduced stability of the microbiota” is meant that the microbiota diversity does not remain as stable and also the relative numbers of the different genera in the microbiota do not remain as stable as the stability observed in a healthy subject or in a population of healthy subjects. In some embodiments, inducing stability of the microbiota results in the stability being induced to a similar level as is present in a healthy subject, or in a population of healthy subjects. In some embodiments, inducing stability of the microbiota results in the stability being induced to the same level as is present in a healthy subject, or in a population of healthy subjects.

Similarly, the invention provides a method of treating or preventing a disease or disorder associated with reduced stability of the microbiota wherein the method comprises administering a composition comprising a bacterial strain of the species Bacteroides thetaiotaomicron. For example, the pathogenesis of some diseases or disorders is characterised by reduced stability of the microbiota. Examples of such diseases and disorders are IBS, IBD, diabetes (e.g. type 2 diabetes), allergic diseases, autoimmune diseases and metabolic diseases/disorders. Accordingly, in some embodiments, the invention provides a composition comprising a bacterial strain of the species Bacteroides thetaiotaomicron, for use in a method of treating or preventing a disease or disorder associated with reduced stability of the microbiota, wherein the treatment or prevention comprises inducing stability of the microbiota. In some embodiments, the disease or disorder is selected from IBS, IBD, diabetes (e.g. type 2 diabetes), allergic diseases, autoimmune diseases and metabolic diseases/disorders. In some embodiments, the disease or disorder is IBS or IBD. In some embodiments, the disease or disorder is Crohn's disease. Accordingly, in some embodiments, the invention provides a composition comprising a bacterial strain of the species Bacteroides thetaiotaomicron, for use in a method of treating or preventing IBS or IBD (in particular Crohn's disease), wherein the treatment or prevention comprises inducing stability of the microbiota. In such embodiments, the composition may be administered to a subject having reduced microbiota diversity and/or stability.

In some embodiments, the invention provides a method of treatment or prevention of a disease or disorder associated with a level of microbiota diversity and/or evenness that is reduced relative to the microbiota diversity of a healthy subject or population of healthy subjects wherein the method comprises diagnosing a subject as having a reduced level of microbiota diversity and then if a reduced level of diversity is found to be present, administering a composition comprising a bacterial strain of the species Bacteroides thetaiotaomicron to the subject.

In some embodiments, the invention provides a method of treatment or prevention of a disease or disorder associated with reduced stability of microbiota relative to the stability of microbiota in a healthy subject wherein the method comprises diagnosing a subject as having reduced stability of microbiota and then if reduced stability is found to be present, administering a composition comprising a bacterial strain of the species Bacteroides thetaiotaomicron to the subject.

Strains closely related to the species Bacteroides thetaiotaomicron may also be used. Such bacterial strains may have a 16s rRNA sequence that is at least 95%, 96%, 97%, 98%, 99%, 99.5% or 99.9% identical to the 16s rRNA sequence of a bacterial strain of Bacteroides thetaiotaomicron. Preferably, the bacterial strain has a 16s rRNA sequence that is at least 95%, 95%, 97%, 98%, 99%, 99.5% or 99.9% identical to any one of SEQ ID NOs:1-12, preferably to SEQ ID NO: 1. Preferably, the bacterial strain has the 16s rRNA sequence of SEQ ID NO:1. Most preferably, the bacterial strain in the composition is the Bacteroides thetaiotaomicron strain deposited under accession number NCIMB 42341.

In certain embodiments, the composition of the invention is for oral administration. Oral administration of the strains of the invention can be effective for increasing the microbiota diversity and/or inducing the stability of the microbiota. Also, oral administration is convenient for subjects and practitioners and allows delivery to and/or partial or total colonisation of the intestine.

In certain embodiments, the composition of the invention comprises one or more pharmaceutically acceptable excipients or carriers.

In certain embodiments, the composition of the invention comprises a bacterial strain that has been lyophilised. Lyophilisation is an effective and convenient technique for preparing stable compositions that allow delivery of bacteria, and is shown to provide effective compositions in the examples.

In certain embodiments, the invention provides a food product comprising the composition as described above.

In certain embodiments, the invention provides a vaccine composition comprising the composition as described above.

Additionally, the invention provides a method of increasing the microbiota diversity and/or inducing the stability of the microbiota and thereby treating or preventing diseases or disorders associated with a reduced microbiota diversity and/or with reduced stability of the microbiota, comprising administering a composition comprising a bacterial strain of the genus Bacteroides.

### BRIEF DESCRIPTION OF DRAWINGS

FIG. 1: Effect of Thetanix treatment on microbiota diversity using Observed Species and Shannon Diversity Metrics

FIG. 2: Effect of Thetanix on microbiota evenness

### DISCLOSURE OF THE INVENTION

### Bacterial Strains

The compositions of the invention comprise a bacterial strain of the genus Bacteroides. The examples demonstrate that bacteria of this genus are useful for increasing the microbiota diversity and/or inducing the stability of the microbiota. The preferred bacterial strains are of the species Bacteroides thetaiotaomicron, particularly the bacterium deposited under accession number NCIMB 42341. Bacteroides is a genus of gram-negative, obligate anaerobic bacteria. Bacteroides species are non endospore-forming bacilli, and may be either motile or nonmotile, depending on the species.

Bacteroides thetaiotaomicron was first described in 1912 under the name Bacillus thetaiotaomicron and moved to the genus Bacteroides in 1919. It was originally isolated from adult human feces. Bacteroides thetaiotaomicron triggers the nuclear export of the RelA subunit of nuclear kappa-light-chain-enhancer of activated B cells (NK-B), an important nuclear transcription factor, thereby limiting the transcription of downstream pro-inflammatory genes and synthesis of inflammatory factors, including interleukin (IL)-9 and tumor necrosis factor alpha (TNFα).

Bacterial strains closely related to the strain tested in the examples are also expected to be effective for increasing the microbiota diversity and/or inducing the stability of the microbiota. In certain embodiments, the bacterial strain for use in the invention has a 16s rRNA sequence that is at least 95%, 96%, 97%, 98%, 99%, 99.5% or 99.9% identical to the 16s rRNA sequence of a bacterial strain of Bacteroides thetaiotaomicron. Preferably, the bacterial strain for use in the invention has a 16s rRNA sequence that is at least 95%, 96%, 97%, 98%, 99%, 99.5% or 99.9% identical to SEQ ID NO:1. Preferably, the bacterial strain for use in the invention has a 16s rRNA sequence that has the sequence of SEQ ID NO:1. Preferably, the bacterial strain for use in the invention belongs to the genus Bacteroides.

Bacterial strains that are biotypes of the bacterium deposited under accession number NCIMB 42341 are also expected to be effective for increasing the microbiota diversity and/or inducing the stability of the microbiota. A biotype is a closely related strain that has the same or very similar physiological and biochemical characteristics.

Strains that are biotypes of a bacterium deposited under accession number NCIMB 42341 and that are suitable for use in the invention may be identified by sequencing other nucleotide sequences for a bacterium deposited under accession number NCIMB 42341. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). For example, in some embodiments, a biotype strain has at least 98% sequence identity across at least 98% of its genome or at least 99% sequence identity across 99% of its genome. Other suitable sequences for use in identifying biotype strains may include hsp60 or repetitive sequences such as BOX, ERIC, (GTG)₅, or REP or [27]. Biotype strains may have sequences with at least 97%, 98%, 99%, 99.5% or 99.9% sequence identity to the corresponding sequence of a bacterium deposited under accession number NCIMB 42341. In some embodiments, a biotype strain has a sequence with at least 97%, 98%, 99%, 99.5% or 99.9% sequence identity to the corresponding sequence of the Bacteroides thetaiotaomicron strain deposited under accession number NCIMB 42341 and comprises a 16S rRNA sequence that is at least 99% identical (e.g. at least 99.5% or at least 99.9% identical) to SEQ ID NO:1. In some embodiments, a biotype strain has a sequence with at least 97%, 98%, 99%, 99.5% or 99.9% sequence identity to the corresponding sequence of the Bacteroides thetaiotaomicron strain deposited under accession number NCIMB 42341 and has the 16S rRNA sequence of SEQ ID NO:1.

Alternatively, strains that are biotypes of a bacterium deposited under accession number NCIMB 42341 and that are suitable for use in the invention may be identified by using the accession number NCIMB 42341 deposit, and restriction fragment analysis and/or PCR analysis, for example by using fluorescent amplified fragment length polymorphism (FAFLP) and repetitive DNA element (rep)-PCR fingerprinting, or protein profiling, or partial 16S or 23s rDNA sequencing. In preferred embodiments, such techniques may be used to identify other Bacteroides thetaiotaomicron strains.

In certain embodiments, strains that are biotypes of a bacterium deposited under accession number NCIMB 42341 and that are suitable for use in the invention are strains that provide the same pattern as a bacterium deposited under accession number NCIMB 42341 when analysed by amplified ribosomal DNA restriction analysis (ARDRA), for example when using Sau3AI restriction enzyme (for exemplary methods and guidance see, for example [28]). Alternatively, biotype strains are identified as strains that have the same carbohydrate fermentation patterns as a bacterium deposited under accession number NCIMB 42341.

Other Bacteroides species that are useful in the compositions and methods of the invention, such as biotypes of a bacterium deposited under accession number NCIMB 42341, may be identified using any appropriate method or strategy. For instance, strains for use in the invention may be identified by culturing bacteria and administering to rats to test in the distension assay. In particular, bacterial strains that have similar growth patterns, metabolic type and/or surface antigens to a bacterium deposited under accession number NCIMB 42341 may be useful in the invention. A useful strain will have comparable microbiota modulatory activity to the NCIMB 42341 strain. In particular, a biotype strain will elicit comparable effects on the microbiota to the effects shown in the Examples.

A particularly preferred strain of the invention is the Bacteroides thetaiotaomicron strain deposited under accession number NCIMB 42341. This is the exemplary strain tested in the examples and shown to be effective for increasing the microbiota diversity and/or inducing the stability of the microbiota. Therefore, the invention provides a cell, such as an isolated cell, of the Bacteroides thetaiotaomicron strain deposited under accession number NCIMB 42341, or a derivative thereof, for use in therapy, in particular for the diseases and disorders described herein.

A derivative of the strain may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable microbiota modulatory activity to the original strain. In particular, a derivative strain will elicit comparable effects on the microbiota to the effects shown in the Examples, which may be identified by using the culturing and administration protocols described in the Examples. A derivative of the NCIMB 42341 strain will generally be a biotype of the NCIMB 42341 strain.

References to cells of the Bacteroides thetaiotaomicron strain deposited under accession number NCIMB 42341 encompass any cells that have the same safety and therapeutic efficacy characteristics as the strains deposited under accession number NCIMB 42341, and such cells are encompassed by the invention.

In preferred embodiments, the bacterial strains in the compositions of the invention are viable and capable of partially or totally colonising the intestine.

### Therapeutic Uses

In certain embodiments, the compositions of the invention are for use in increasing the microbiota diversity, evenness and/or inducing the stability of the microbiota. Reduced diversity or evenness of the microbiota and/or reduced stability of the microbiota are associated with numerous pathological diseases and disorders, as discussed above, and the examples demonstrate that the compositions of the invention may be effective for increasing the microbiota diversity and evenness and/or inducing the stability of the microbiota. Accordingly, the disease or disorder to be treated or prevented using a composition of the invention is preferably a disease or disorder associated with a level of microbiota diversity and/or evenness that is reduced relative to the microbiota diversity and/or evenness of a healthy subject and/or a disease or disorder that is associated with reduced stability of the microbiota. Thus, in some embodiments, the disease or disorder may be associated with a level of microbiota diversity and/or evenness that is reduced relative to the microbiota diversity of a healthy subject and also be associated with reduced stability of the microbiota.

In certain embodiments, the compositions of the invention are for use in increasing the microbiota diversity, evenness and/or inducing the stability of the microbiota in patients diagnosed with a disease or disorder selected from IBS, IBD (including Crohn's disease), cancer (including colorectal cancer) optionally in patients receiving concomitant anti-cancer therapies such as chemotherapy, obesity, type 2 diabetes, one or more infectious diseases, one or more allergic diseases, one or more autoimmune diseases and one or more metabolic diseases/disorders. Use of the compositions of the invention to increase the microbiota diversity, evenness and/or induce the stability of the microbiota in patients diagnosed with other diseases and disorders is also envisaged. In certain embodiments, the compositions of the invention are for use in treating or preventing IBS or IBD. In certain embodiments, the compositions of the invention are for use in treating or preventing IBS. In certain embodiments, the compositions of the invention are for use in treating or preventing IBD. In certain embodiments, the compositions of the invention are for use in treating or preventing one or more allergic diseases. In certain embodiments, the compositions of the invention are for use in treating or preventing cancer optionally in patients administered concomitant anticancer therapy. In certain embodiments, the compositions of the invention are for use in treating or preventing obesity. In certain embodiments, the compositions of the invention are for use in treating or preventing one or more infectious diseases. In certain embodiments, the compositions of the invention are for use in treating or preventing one or more autoimmune diseases. In certain embodiments, the compositions of the invention are for use in treating or preventing one or more metabolic diseases/disorders. Preferably, the treatment or prevention comprises increasing the microbiota diversity and/or inducing the stability of the microbiota in the subject. Preferably the disease which is treated is Crohn's disease.

In certain embodiments, the one or more infectious diseases is selected from a viral, bacterial or fungal disease. In certain embodiments, the one or more allergic diseases is asthma. In certain embodiments, the one or more metabolic diseases/disorders is selected from diabetes, e.g. type 2 diabetes, and obesity. In certain embodiments, the one or more autoimmune diseases is selected from multiple sclerosis and rheumatoid arthritis.

In certain embodiments, the compositions of the invention are for use in treating or preventing IBS, IBD (including Crohn's disease), obesity, type 2 diabetes, one of more infectious diseases, one or more allergic diseases, one or more autoimmune diseases or one or more metabolic diseases/disorders by increasing the microbiota diversity in the microbiota. In certain embodiments, the compositions of the invention are for use in treating or preventing IBS or IBD by inducing the stability of the microbiota. In certain embodiments, the compositions of the invention are for use in treating or preventing IBD by inducing the stability of the microbiota

In preferred embodiments, the invention provides a composition comprising a bacterial strain of the species Bacteroides thetaiotaomicron, for use in the treatment or prevention of IBD, IBS, obesity, type 2 diabetes, one or more infectious diseases, one or more allergic diseases, one or more autoimmune diseases or one or more metabolic diseases/disorders, wherein the treatment or prevention comprises increasing the microbiota diversity and/or inducing the stability of the microbiota in the subject.

In some embodiments, the invention provides a composition comprising a bacterial strain of the species Bacteroides thetaiotaomicron for use in treating or preventing a disease or disorder selected from IBS, IBD, obesity, type 2 diabetes, one or more infectious diseases, one or more allergic diseases, one or more autoimmune diseases and one or more metabolic diseases/disorders. In some embodiments, the invention provides a method of treating or preventing a disease or disorder selected from IBS, IBD, obesity, type 2 diabetes, one or more infectious diseases, one or more allergic diseases, one or more autoimmune diseases and one or more metabolic diseases/disorders, comprising administering a composition comprising a bacterial strain of the species Bacteroides thetaiotaomicron.

In preferred embodiments, the compositions of the invention comprise the bacterium deposited under accession number NCIMB 42341 and are for use in increasing the microbiota diversity and/or inducing the stability of the microbiota in the subject in the treatment of IBD, IBS, obesity, type 2 diabetes, one or more infectious diseases, one or more allergic diseases, one or more autoimmune diseases or one or more metabolic diseases/disorders. In further preferred embodiments, the compositions of the invention comprise the bacterium deposited under accession number NCIMB 42341 and are for use in treating or preventing IBD, IBS, obesity, type 2 diabetes, one or more infectious diseases, one or more allergic diseases, one or more autoimmune diseases or one or more metabolic diseases/disorders by increasing the microbiota diversity and/or inducing the stability of the microbiota.

In some embodiments, the pathogenesis of the disease or disorder affects the intestine. In some embodiments, the pathogenesis of the disease or disorder does not affect the intestine. In some embodiments, the pathogenesis of the disease or disorder is not localised at the intestine. In some embodiments, the treating or preventing occurs at a site other than at the intestine. In some embodiments, the treating or preventing occurs at the intestine and also at a site other than at the intestine. In certain embodiments, the disease or disorder is systemic.

In certain embodiments, the compositions are for use in subjects that exhibit, or are expected to exhibit, reduced levels of microbiota diversity, for example, when compared to a healthy subject, or a population of healthy subjects. For example, in some embodiments, the composition is for use in treating a subject having less than 101 different bacterial species (e.g. less than 100, 99, 98, 97, 96, 95, 93, 90, 85, 80, 75 or 70 bacterial species) and/or less than 195 different strains (e.g. less than 193, 190, 187, 185, 183, 180, 175, 170, 165, 160, 150, 140 bacterial strains) in its microbiota. For example, in some embodiments, the composition is for use in treating a subject that has at least one bacterial genus (e.g. at least 2, 3, 4, 5, 6, 7, 8, 9 or 10 bacterial genera) fewer in its intestinal microbiota compared to a healthy subject or compared to a population of healthy subjects. In some embodiments, the treatment or prevention comprises a step of diagnosing a subject as having a reduced level of microbiota diversity and then if a reduced level of diversity is found to be present, the subject is then treated with a composition according to the invention.

In certain embodiments, the compositions are for use in subjects that exhibit, or are expected to exhibit, reduced stability of the microbiota. In some embodiments, the compositions are for use in subjects that exhibit, or are expected to exhibit, reduced stability in its microbiota, for example, when compared to a healthy subject, or a population of healthy subjects. In some embodiments, the treatment or prevention comprises a step of diagnosing a subject as having a reduced stability in its microbiota and then if reduced stability is found to be present, the subject is then treated with a composition according to the invention.

In certain embodiments, the subject is an infant. In certain embodiments, the subject is a child. In certain embodiments, the subject is an adult. The subject may be an adolescent, for example a subject with an age between 10 and 19 years.

In certain embodiments, the subject is a healthy subject. For example, in some embodiments in which the composition is used for preventing a disease or disorder, the subject is a healthy subject, optionally one identified as being at risk of developing a disease or disorder characterised by a reduction in microbiota diversity.

In certain embodiments, the subject has previously received, is receiving, or will be receiving anticancer treatment, for example chemotherapy. Accordingly, in some embodiments, the treatment or prevention comprises administering the composition of the invention after, together with, or before anticancer treatment.

In certain embodiments, the subject has previously received, is receiving, or will be receiving antibiotic treatment. Accordingly, in some embodiments, the treatment or prevention comprises administering the composition of the invention after, together with, or before antibiotic treatment. The composition of the invention and the one or more antibiotics may be for separate, simultaneous or sequential administration.

Treatment or prevention may refer to, for example, an alleviation of the severity of symptoms or a reduction in the frequency of exacerbations or the range of triggers that are a problem for the subject.

Bacteria in the microbiota may be detected in faeces from a subject, using standard techniques, such as the qPCR techniques used in the examples.

### Modes of Administration

Preferably, the compositions of the invention are to be administered to the gastrointestinal tract in order to enable delivery to and/or partial or total colonisation of the intestine with the bacterial strain of the invention. Generally, the compositions of the invention are administered orally, but they may be administered rectally, intranasally, or via buccal or sublingual routes.

In certain embodiments, the compositions of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily. The examples demonstrate that daily administration provides successful delivery and clinical benefits.

In certain embodiments, the compositions of the invention are administered regularly, such as daily, every two days, or weekly, for an extended period of time, such as for at least one week, two weeks, one month, two months, six months, or one year.

In certain embodiments of the invention, treatment according to the invention is accompanied by assessment of the subject's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to prevent reduced levels of diversity in the microbiota and/or reduced stability of the microbiota developing in her child in utero and/or after it is born.

The compositions of the invention may be administered to a subject that has been diagnosed with reduced microbiota diversity relative to a healthy subject and/or reduced stability of the microbiota or a disease or disorder associated with reduced microbiota diversity relative to a healthy subject and/or reduced stability of the microbiota, or that has been identified as being at risk of reduced microbiota diversity relative to a healthy subject and/or reduced stability of the microbiota. The compositions may also be administered as a prophylactic measure to prevent the development of reduced microbiota diversity relative to a healthy subject and/or reduced stability of the microbiota in a healthy subject.

The compositions of the invention may be administered to a subject that has been identified as having an abnormal gut microbiota. For example, the subject may have reduced or absent colonisation by Bacteroides, and in particular Bacteroides thetaiotaomicron.

The compositions of the invention may be administered as a food product, such as a nutritional supplement.

Generally, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

### Compositions

Generally, the composition of the invention comprises bacteria. In preferred embodiments of the invention, the composition is formulated in freeze-dried form. For example, the composition of the invention may comprise granules or gelatin capsules, for example hard gelatin capsules, comprising a bacterial strain of the invention.

Preferably, the composition of the invention comprises lyophilised bacteria. Lyophilisation of bacteria is a well-established procedure and relevant guidance is available in, for example, references [29-31]. The examples demonstrate that lyophilisate compositions are particularly effective.

Alternatively, the composition of the invention may comprise a live, active bacterial culture.

In some embodiments, the bacterial strain in the composition of the invention has not been inactivated, for example, has not been heat-inactivated. In some embodiments, the bacterial strain in the composition of the invention has not been killed, for example, has not been heat-killed. In some embodiments, the bacterial strain in the composition of the invention has not been attenuated, for example, has not been heat-attenuated. For example, in some embodiments, the bacterial strain in the composition of the invention has not been killed, inactivated and/or attenuated. For example, in some embodiments, the bacterial strain in the composition of the invention is live. For example, in some embodiments, the bacterial strain in the composition of the invention is viable. For example, in some embodiments, the bacterial strain in the composition of the invention is capable of partially or totally colonising the intestine. For example, in some embodiments, the bacterial strain in the composition of the invention is viable and capable of partially or totally colonising the intestine.

In some embodiments, the composition comprises a mixture of live bacterial strains and bacterial strains that have been killed.

In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [32] and [33].

The composition may be administered orally and may be in the form of a tablet, capsule or powder. Encapsulated products are preferred because Blautia are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The composition may be formulated as a probiotic.

A composition of the invention includes a therapeutically effective amount of a bacterial strain of the invention. A therapeutically effective amount of a bacterial strain is sufficient to exert a beneficial effect upon a subject. A therapeutically effective amount of a bacterial strain may be sufficient to result in delivery to and/or partial or total colonisation of the subject's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU; in another example from about 1×10⁶ to about 1×10¹⁰ CFU.

In certain embodiments, the dose of the bacteria is at least 10⁹ cells per day, such as at least 10¹⁰, at least 10¹¹, or at least 10¹² cells per day.

In certain embodiments, the composition contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g. In preferred embodiments, the composition contains the bacterial strain in an amount from about 1×10⁶ to about 1×10^{9.5}.

Typically, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In certain embodiments, the probiotic composition of the present invention includes a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The compositions of the invention may comprise pharmaceutically acceptable excipients or carriers. Examples of such suitable excipients may be found in the reference [34]. Acceptable carriers or diluents for therapeutic use are well known in the pharmaceutical art and are described, for example, in reference [35]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid, cysteine and esters of p-hydroxybenzoic acid. Antioxidants and suspending agents may be also used. A further example of a suitable carrier is saccharose. A further example of a preservative is cysteine.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture or lyophilisate that is substantially free from other species of organism.

In certain embodiments, the compositions of the invention comprise one or more bacterial strains of the genus Bacteroides and do not contain any other bacterial genera, or which comprise only de minimis or biologically irrelevant amounts of bacteria from another genus. In certain embodiments, the compositions of the invention comprise a single species of Bacteroides, preferably Bacteroides thetaiotaomicron, and do not contain any other bacterial species, or which comprise only de minimis or biologically irrelevant amounts of bacteria from another species. In certain embodiments, the compositions of the invention comprise a single strain of Bacteroides, for example, of Bacteroides thetaiotaomicron NCIMB 42341 and do not contain any other bacterial strains or species, or which comprise only de minimis or biologically irrelevant amounts of bacteria from another strain or species.

In some embodiments, the compositions of the invention comprise more than one bacterial strain or species. For example, in some embodiments, the compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. In some embodiments, the compositions of the invention comprise more than one species from within the same genus (e.g. more than 1, 2, 3, 4, 5, 6, 7, 8, 9, 10, 12, 15, 17, 20, 23, 25, 30, 35 or 40 species), and, optionally, do not contain bacteria from any other genus. In some embodiments, the compositions of the invention comprise less than 50 species from within the same genus (e.g. less than 50, 45, 40, 35, 30, 25, 20, 15, 12, 10, 8, 7, 6, 5, 4 or 3 species), and, optionally, do not contain bacteria from any other genus. In some embodiments, the compositions of the invention comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 species from within the same genus and, optionally, do not contain bacteria from any other genus. The invention comprises any combination of the foregoing.

In some embodiments, the composition comprises a microbial consortium. For example, in some embodiments, the composition comprises the Bacteroides bacterial strain, for example, a Bacteroides thetaiotaomicron bacterial strain as part of a microbial consortium. For example, in some embodiments, the Bacteroides bacterial strain is present in combination with one or more (e.g. at least 2, 3, 4, 5, 10, 15 or 20) other bacterial strains from other genera with which it can live symbiotically in vivo in the intestine. For example, in some embodiments, the composition comprises a bacterial strain of Bacteroides thetaiotaomicron in combination with a bacterial strain from a different genus. In some embodiments, the microbial consortium comprises two or more bacterial strains obtained from a faeces sample of a single organism, e.g. a human. In some embodiments, the microbial consortium is not found together in nature. For example, in some embodiments, the microbial consortium comprises bacterial strains obtained from faeces samples of at least two different organisms. In some embodiments, the two different organisms are from the same species, e.g. two different humans. In some embodiments, the two different organisms are an infant human and an adult human. In some embodiments, the two different organisms are a human and a non-human mammal.

In some embodiments, the composition of the invention additionally comprises a bacterial strain that has the same safety and therapeutic efficacy characteristics as the Bacteroides thetaiotaomicron strain deposited under accession number NCIMB 42341, but which is not the Bacteroides thetaiotaomicron strain deposited under accession number NCIMB 42341, or which is not a Bacteroides thetaiotaomicron strain.

In some embodiments in which the composition of the invention comprises more than one bacterial strain, species or genus, the individual bacterial strains, species or genera may be for separate, simultaneous or sequential administration. For example, the composition may comprise all of the more than one bacterial strains, species or genera, or the bacterial strains, species or genera may be stored separately and be administered separately, simultaneously or sequentially. In some embodiments, the more than one bacterial strains, species or genera are stored separately but are mixed together prior to use.

In some embodiments, the bacterial strain for use in the invention is obtained from human adult faeces. In some embodiments in which the composition of the invention comprises more than one bacterial strain, all of the bacterial strains are obtained from human adult faeces or if other bacterial strains are present they are present only in de minimis amounts. In some embodiments, the bacteria may have been cultured subsequent to being obtained from the human adult faeces and being used in a composition of the invention.

In some embodiments, the one or more Bacteroides bacterial strains (for example the Bacteroides thetaiotaomicron strain) is/are the only therapeutically active agent(s) in a composition of the invention. In some embodiments, the bacterial strain(s) in the composition is/are the only therapeutically active agent(s) in a composition of the invention.

The compositions for use in accordance with the invention may or may not require marketing approval.

In certain embodiments, the invention provides the above pharmaceutical composition, wherein said bacterial strain is lyophilised. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is lyophilised or spray dried and wherein it is viable and capable of partially or totally colonising the intestine.

In some cases, the lyophilised or spray dried bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain as used in the invention; and a pharmaceutically acceptable excipient, carrier or diluent; wherein the bacterial strain is in an amount sufficient to increase the microbiota diversity in a subject and/or induce stability of the microbiota and/or treat a disorder associated with reduced microbiota diversity and/or reduced stability of the microbiota when administered to a subject in need thereof, the disorder associated with microbiota diversity being selected from, for example, IBS, IBD, cancer, obesity, type 2 diabetes, one or more infectious diseases, one or more allergic diseases, one or more autoimmune diseases or one or more metabolic diseases/disorders.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilised.

In certain embodiments, the invention provides the above pharmaceutical composition, wherein when the composition is stored in a sealed container at about 4° C. or about 25° C. and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”).

In some embodiments, the compositions of the invention are administered orally. Oral administration may involve swallowing, so that the compound enters the gastrointestinal tract, and/or buccal, lingual, or sublingual administration by which the compound enters the blood stream directly from the mouth.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions.

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film forming polymer (e.g. see [36]). In some embodiments, the formulation is an intrinsically enteric capsule (for example, Vcaps® from Capsugel).

In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

### Culturing Methods

The bacterial strains for use in the present invention can be cultured using standard microbiology techniques as detailed in, for example, references [37-39].

The solid or liquid medium used for culture may be YCFA agar or YCFA medium. YCFA medium may include (per 100 ml, approximate values): Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄·7H₂O (0.009 g), CaCl₂) (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

### Bacterial Strains for Use in Vaccine Compositions

The inventors have identified that the bacterial strains of the invention are useful for treating or preventing diseases or disorders associated with a level of microbiota diversity that is reduced relative to the microbiota diversity of a healthy subject (or relative to the microbiota diversity of a population of healthy subjects) and/or diseases or disorders that are associated with reduced stability of the microbiota compared to a healthy subject (or compared to a population of healthy subjects). This is likely to be a result of the effect that the bacterial strains of the invention have on the host immune system. Therefore, the compositions of the invention may also be useful for preventing such diseases or disorders when administered as vaccine compositions. These vaccines comprise a B. thetaiotaomicron antigen. In certain such embodiments, the bacterial strains of the invention are viable. In certain such embodiments, the bacterial strains of the invention are capable of partially or totally colonising the intestine. In certain such embodiments, the bacterial strains of the invention are viable and capable of partially or totally colonising the intestine. In other certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references [40] and [41-47], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [48]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. [49].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### MODES FOR CARRYING OUT THE INVENTION

### Example 1—Effect of Thetanix on Microbiota Diversity

Thetanix is a live biotherapeutic containing the bacterium Bacteroides thetaiotaomicron (B. Theta) as the active ingredient. It is lyophilised and formulated as gastro-resistant capsules for oral administration. Each capsule contains 10^{7.73±1.43} colony forming units (CFUs).

### Overall Study Design

The study was a randomised, double-blind, placebo-controlled, multiple dose study in subjects aged 16 to 18 years with Crohn's disease. Subjects suitable for the study were identified from patient lists at appropriate gastroenterology clinics.

The patients received daily dosing over 7.5 days where the first dose was taken on Day 0 (D0) in clinic, the next 13 doses were taken at home and the 15th dose was taken in the clinic. Subject received a dose of B. Theta or placebo an hour before food every 12 hours during the 7.5 day dosing period.

Stool samples were collected at D0, D1, D7 and D56. These were analysed by quantitative polymerase chain reaction (PCR) for B. theta and other common constituents of the microbiome.

### Results

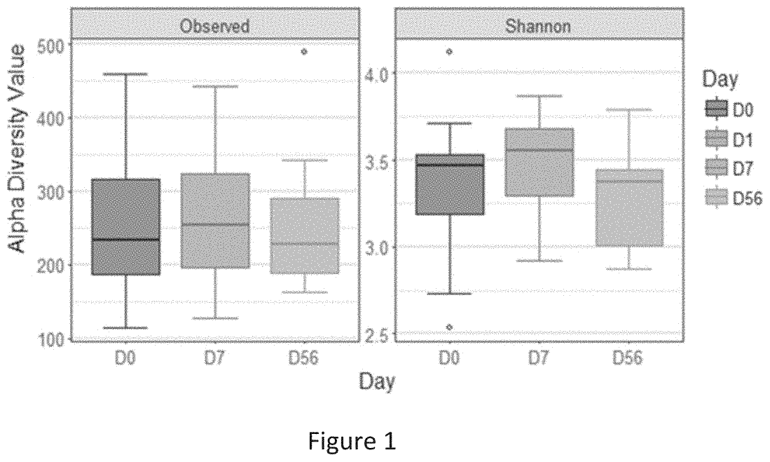

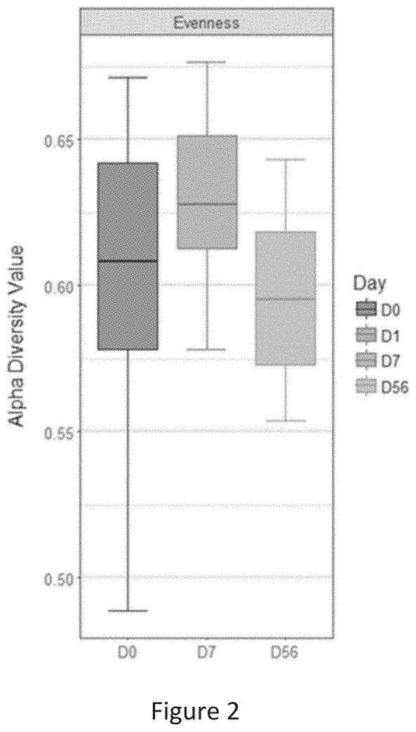

The effect of treatment on microbiota diversity was assessed using the number of Observed species per sample (richness) and the Shannon Diversity Index which represents the number of taxa (richness) and their relative abundances (evenness) within each sample. The effects of Thetanix treatment on microbiota diversity are shown in FIG. 1 which shows a significant difference in Shannon Diversity between the study timepoints (D0, D7 and D56). Similarly, microbiota evenness was found to be significant across the study timepoints, as shown in FIG. 2.

### CONCLUSIONS

B. Theta was well tolerated in the study. There were no serious adverse events, deaths or subjects who discontinued from the study after treatment. There were no trends in haematology, clinical chemistry, vital signs, or physical examinations to suggest an adverse effect of B. Theta on these parameters.

Although the study was conducted in a small population, Thetanix shows promise as an agent capable of increasing diversity and evenness in the microbiota. Given the association between disease and a loss of microbiota diversity, Thetanix can be expected to treat conditions like Crohn's disease which are associated with reduced microbiome diversity.

Furthermore, a significant change in the faecal calprotectin levels was observed in several of the patients administered Thetanix over the course of the study indicating the efficacy of Thetanix treatment in Crohn's disease.

The invention has been described above by way of example only and it will be understood that further modifications may be made which fall within the scope of the claims.

Sequences
(Bacteroides thetaiotaomicron strain NCIMB 42341 16S ribosomal RNA gene)
SEQ ID NO: 1
cttttacaat gaagagtttg atcctggctc aggatgaacg ctagctacag gcttaacaca 60
tgcaagtcga ggggcagcat ttcagtttgc ttgcaaactg gagatggcga ccggcgcacg 120
ggtgagtaac acgtatccaa cctgccgata actcggggat agcctttcga aagaaagatt 180
aatacccgat ggtataatca gaccgcatgg tttgattatt aaagaatttc gcttatcgat 240
ggggatgcgt tccattaggc agttggtgag gtaacggctc accaaacctt cgatggatag 300
gggttctgag aggaaggtcc cccacattgg aactgagaca cggtccaaac tcctacggga 360
ggcagcagtg aggaatattg gtcaatgggc gcaggcctga accagccaag tagcgtgaag 420
gatgactgcc ctatgggttg taaacttctt ttatatggga ataaagtttt ccacgtgtgg 480
aattttgtat gtaccatatg aataaggatc ggctaactcc gtgccagcag ccgcggtaat 540
acggaggatc cgagcgttat ccggatttat tgggtttaaa gggagcgtag gtggacagtt 600
aagtcagttg tgaaagtttg cggctcaacc gtaaaattgc agttgatact ggctgtcttg 660
agtacagtag aggtgggcgg aattcgtggt gtagcggtga aatgcttaga tatcacgaag 720
aactccgatt gcgaaggcag ctcactggac tgcaactgac actgatgctc gaaagtgtgg 780
gtatcaaaca ggattagata ccctggtagt ccacacagta aacgatgaat actcgctgtt 840
tgcgatatac agtaagcggc caagcgaaag cattaagtat tccacctggg gagtacgccg 900
gcaacggtga aactcaaagg aattgacggg ggcccgcaca agcggaggaa catgtggttt 960
aattcgatga tacgcgagga accttacccg ggcttaaatt gcatttgaat atattggaaa 1020
cagtatagcc gtaaggcaaa tgtgaaggtg ctgcatggtt gtcgtcagct cgtgccgtga 1080
ggtgtcggct taagtgccat aacgagcgca acccttatct ttagttacta acaggtcatg 1140
ctgaggactc tagagagact gccgtcgtaa gatgtgagga aggtggggat gacgtcaaat 1200
cagcacggcc cttacgtccg gggctacaca cgtgttacaa tggggggtac agaaggcagc 1260
tacctggtga caggatgcta atcccaaaag cctctctcag ttcggatcga agtctgcaac 1320
ccgacttcgt gaagctggat tcgctagtaa tcgcgcatca gccatggcgc ggtgaatacg 1380
ttcccgggcc ttgtacacac cgcccgtcaa gccatgaaag ccgggggtac ctgaagtacg 1440
taaccgcaag gagcgtccta gggtaaaact ggtaattggg gc 1482
(Bacteroides thetaiotaomicron (ATCC 29148) 16S rRNA)
SEQ ID NO: 2
cantgaagag tttgatcctg gctcaggatn aacgctagct acaggcttaa cacatgcaag 60
tcgaggggca gcatttcnnt ttgcttgcaa actnnagatg gcgaccggcg cacgggtgag 120
taacacgtat ccaacctgcc gataactcgg ggatagcctt tcgaaagaaa gattaatacc 180
cgatggcata atcanaccgc atggtcttat tattaaagaa tttcggttat cgatggggat 240
gcgttccatt aggcagttgg tgaggtaacg gctcacnaaa ccttcgatgg ataggggttc 300
tgagaggaag gtcccccaca ttggaactga gacacggtcc naactcctac gggaggcagc 360
agtgaggaat attggtcaat gggcgcaggc ctnaaccagc caagtagcgt gaaggatgac 420
tgccctatgg gttgtaaact nctnttatat gggaataaag tnttccacgt gtggaatttt 480
gtatgtacca tatgaataag gatcggctaa ctccgtgcca gcagccgcgg tnatacggag 540
gatccgagcg ttatccggat ttattgggtt taaagggagc gtaggtggac agttaagtca 600
gttgtgaaag tttgcggctc aaccgtaaaa ttgcagttga tactggctgt cttgagtaca 660
gtagaggtgg gcggaattcg tggtgtagcg gtgaaatgct tagatatcac gaagaactcc 720
gattgcgaag gcagctcact ggactgcaac tgacactgat gctcgaaagt gtgggtatca 780
aacaggatta gataccctgg tagtccacac agtaaacgat gaatactcgc tctttgcgat 840
atacagtaag cggccaagcg aaagcattaa gtattccacc tggggagtac gccggcaacg 900
gtgaaactca aaggaattga cgggggcccg cacaagcgga ggaacatgtg gtttaattcg 960
atgatacgcg aggaacctta cccgggctta aattgcattt gaataatctg gaaacaggtt 1020
agccgcaagg caaatgtgaa ggtgctgcat ggttgtcgtc agctcgtgcc gtgaggtgtc 1080
ggcttaagtg ccataacgag cccaaccctt atctttagtt actaacaggt catgctgagg 1140
actctagaga gactgccgtc gtaagatgtg aggaaggtgg ggatgacgtc aaatcagcac 1200
ggcccttacg tccggggcta cacacgtgtt acaatggggg gtacagaagg cagctacctg 1260
gtgacaggat gctnatccca aaagcctctc tcagttcgga tcgaagtctg caacccgact 1320
tcgtgaagct ggattcgcta gtaatcgcgc atcagccatg gcgcggtgaa tacgttcccg 1380
ggccttgtac acaccgcccg tcaanccatg anagccgggg gtacctgaag tacgtaaccg 1440
caaggagcgt cctagggtaa aactggtaat tgggg 1475
(Bacteroides thetaiotaomicron strain WAL 2926 (M58763) 16S rRNA)
SEQ ID NO: 3
cttntacaat gaagagtttg atcctggctc aggatnaacg ctagctacag gcttaacaca 60
tgcaagtcna ggggcagcat ttcagtttgc ttgcaaactg gagatggcga ccggcgcacg 120
ggtgagtaac acgtatccaa cctgccgata actcggggat agcctttcga aagaaagatt 180
aatacccnat ggtataatca gaccgcatng tcttrttatt aaagaatttc gcttatcgat 240
ggggatgcgt tccattaggc agttggtgag gtaacggctc acnnaacctt cgatggatag 300
gggttctgag aggaaggtcc cccacattgg aactgagaca cggtccaaac tcctacggga 360
ggcagcagtg aggaatattg gtcaatgggc gcaggcctga accagccaag tagcgtgaag 420
gatgactgcc ctatgggttg taaacttctt ttatatggga ataaagtttt ccacgtgtgg 480
aattttgtat gtaccatatg aataaggatc ggctaactcc gtgccagcag ccncgntnat 540
acggagnatc cgagcgttat ccggatttat tcggtttaaa gggagcgtag gtggacagtt 600
aagtcagttg tgaaagtttg cggctcaacc gtaaaattgc agttgatact ggctgtcttg 660
agtacagtag aggtgggcgg aattcgtggt gtagcggtga aatgcttaga tatcacgaag 720
aactccgatt gcgaaggcag ctcactggac tgcaactgac actgatgctc gaaagtgtgg 780
gtatcaaaca ggattagata ccctggtagt ccacacagta aacgatgaat actcgctgtt 840
tgcgatatac agtaagcggc caagcgaaag cattaagtat tccacctggg gagtacgccg 900
gcaacggtga aactcaaagg aattgacggg ggccngcaca agcggaggaa catgtggttt 960
aattcgatga tacgcgagga accttacccg ggcttaaatt gcatttgaat atattggaaa 1020
cagtatagcc gyaaggcaaa tgtgaaggtg ctgcatggtt gtcgtcagct cgtgccgtga 1080
ggtgtcggct taagtgccat aacgagcgca acccttatct ttagttacta acaggtcatg 1140
ctgaggactc tagagagact gccgtcgtaa gatgtgagga aggtggggat gacgtcaaat 1200
cagcacngcc cntacgtccg gggctacaca cgtgttacaa tggggggtac agaaggcagc 1260
tacctggtga caggatgcta atcccaaaag cctctctcag ttcggatcga agtctgcaac 1320
ccgacttcgt gaagctggat tcgctagtaa tcgcgcatca gccatggcgc ggtgaatacg 1380
ttcccgggcn ttgtacacac cgcccgtcaa gccatgaaag ccgggggtac ctgaagtacg 1440
taaccgcaag gagcgtccta gggtaaaact ggtaattggg gc 1482
(Bacteroides thetaiotaomicron gene for 16S rRNA-BT-A)
SEQ ID NO: 4
gttttcccta ggacgctcct tgcggttacg tacttcaggt acccccggct ttcatggctt 60
gacgggcggt gtgtacaagg cccgggaacg tattcaccgc gccatggctg atgcgcgatt 120
actagcgaat ccagcttcac gaagtcgggt tgcagacttc gatccgaact gagagaggct 180
tttgggatta gcatcctgtc accaggtagc tgccttctgt accccccatt gtaacacgtg 240
tgtagccccg gacgtaaggg ccgtgctgat ttgacgtcat ccccaccttc ctcacatctt 300
acgacggcag tctctctaga gtcctcagca tgacctgtta gtaactaaag ataagggttg 360
cgctcgttat ggcacttaag ccgacacctc acggcacgag ctgacgacaa ccatgcagca 420
ccttcacatt tgccttacgg ctatactgtt tccaatatat tcaaatgcaa tttaagcccg 480
ggtaaggttc ctcgcgtatc atcgaattaa accacatgtt cctccgcttg tgcgggcccc 540
cgtcaattcc tttgagtttc accgttgccg gcgtactccc caggtggaat acttaatgct 600
ttcgcttggc cgcttactgt atatcgcaaa cagcgagtat tcatcgttta ctgtgtggac 660
taccagggta tctaatcctg tttgataccc acactttcga gcatcagtgt cagttgcagt 720
ccagtgagct gccttcgcaa tcggagttct tcgtgatatc taagcatttc accgctacac 780
cacgaattcc gcccacctct actgtactca agacagccag tatcaactgc aattttacgg 840
ttgagccgca aactttcaca actgacttaa ctgtccacct acgctccctt taaacccaat 900
aaatccggat aacgctcgga tcctccgtat taccgcggct gctggcacgg agttagccga 960
tccttattca tatggtacat acaaaattcc acacgtggaa aactttattc ccatataaaa 1020
gaagtttaca acccataggg cagtcatcct tcacgctact tggctggttc aggcctgcgc 1080
ccattgacca atattcctca ctgctgcctc ccgtaggagt ttggaccgtg tctcagttcc 1140
antgtggggg accttcctct cagaacccct atccatcgaa ggtttggtga gccgttacct 1200
caccaactgc ctaatggaac gcatccccat cgataaccga aattctttaa taacaagacc 1260
atgcggtcta attataccat cggatattaa tctttctttc gaaaggctat ccccgagtta 1320
tcggcaggtt ggatacgtgt tactcacccg tgcgccggtc gccatcttca gttgcaagca 1380
aactgaaatg ctgcccctcg acttgcatgg taagcc 1416
(Bacteroides thetaiotaomicron gene for 16S rRNA-BT-B)
SEQ ID NO: 5
gctccttgcg gttacgtact tcaggtaccc ccggctttca tggcttgacg ggcggtgtgt 60
acaaggcccg ggaacgtatt caccgcgcca tggctgatgc gcgattacta gcgaatccag 120
cttcacgaag tcgggttgca gacttcgatc cgaactgaga gaggcttttg ggattagcat 180
cctgtcacca ggtagctgcc ttctgtaccc cccattgtaa cacgtgtgta gccccggacg 240
taagggccgt gctgatttga cgtcatcccc accttcctca catcttacga cggcagtctc 300
tctagagtcc tcagcataac ctgttagtaa ctaaagataa gggttgcgct cgttatggca 360
cttaagccga cacctcacgg cacgagctga cgacaaccat gcagcacctt cacatttgcc 420
ttgcgactaa cctgtttcca gattattcaa atgcaattta agcccgggta aggttcctcg 480
cgtatcatcg aattaaacca catgttcctc cgcttgtgcg ggcccccgtc aattcctttg 540
agtttcaccg ttgccggcgt actccccagg tggaatactt aatgctttcg cttggccgct 600
tactgtatat cgcaaacagc gagtattcat cgtttactgt gtggactacc agggtatcta 660
atcctgtttg atacccacac tttcgagcat cagtgtcagt tgcagtccag tgagctgcct 720
tcgcaatcgg agttcttcgt gatatctaag catttcaccg ctacaccacg aattccgccc 780
acctctactg tactcaagac agccagtatc aactgcaatt ttacggttga gccgcaaact 840
ttcacaactg acttaactgt ccacctacgc tccctttaaa cccaataaat ccggataacg 900
ctcggatcct ccgtattacc gcggctgctg gcacggagtt agccgatcct tattcatatg 960
gtacatacaa aattccacac gtggaaaact ttattcccat ataaaagaag tttacaaccc 1020
atagggcagt catccttcac gctacttggc tggttcaggc ctgcgcccat tgaccaatat 1080
tcctcactgc tgcctcccgt aggagtttgg accgtgtctc agttccaatg tgggggacct 1140
tcctctcaga acccctatcc atcgaaggtt tggtgagccg ttacctcacc aactgcctaa 1200
tggaacgcat ccccatcgat aaccgaaatt ctttaataac aagaccatgc ggtctaatta 1260
taccatcggg tattaatctt tctttcgaaa ggctatcccc gagttatcgg caggttggat 1320
acgtgttact cacccgtgcg ccggtcgcca tctccagttt gcaagcaaac tgaaatgctg 1380
cccctcgact gca 1393
(Bacteroides thetaiotaomicron gene for 16S rRNA-BT-C)
SEQ ID NO: 6
gctccttgcg gttacgtact tcaggtaccc ccggctttca tggcttgacg ggcggtgtgt 60
acaaggcccg ggaacgtatt caccgcgcca tggctgatgc gcgattacta gcgaatccag 120
cttcacgaag tcgggttgca gacttcgatc cgaactgaga gaggcttttg ggattagcat 180
cctgtcacca ggtagctgcc ttctgtaccc cccattgtaa cacgtgtgta gccccggacg 240
taagggccgt gctgatttga cgtcatcccc accttcctca catcttacga cggcagtctc 300
tctagagtcc tcagcatgac ctgttagtaa ctaaagataa gggttgcgct cgttatggca 360
cttaagccga cacctcacgg cacgagctga cgacaaccat gcagcacctt cacatttgcc 420
ttacggctat actgtttcca gtatattcaa atgcaattta agcccgggta aggttcctcg 480
cgtatcatcg aattaaacca catgttcctc cgcttgtgcg ggcccccgtc aattcctttg 540
agtttcaccg ttgccggcgt actccccagg tggaatactt aatgctttcg cttggccgct 600
tactgtatat cccaaacagc gagtattcat cgtttactgt gtggactacc agggtatcta 660
atcctgtttg atacccacac tttcgagcat cagtgtcagt tgcagtccag tgagctgcct 720
tcgcaatcgg agttcttcgt gatatctaag catttcaccg ctacaccacg aattccgccc 780
acctctactg tactcaagac agccagtatc aactgcaatt ttacggttga gccgcaaact 840
ttcacaactg acttaactgt ccacctacgc tccctttaaa cccaataaat ccggataacg 900
ctcggatcct ccgtattacc gcggctgctg gcacggagtt agccgatcct tattcatatg 960
gtacatacaa aattccacac gtggaaaact ttattcccat ataaaagaag tttacaaccc 1020
atagggcagt catccttcac gctacttggc tggttcaggc ctgcgcccat tgaccaatat 1080
tcctcactgc tgcctcccgt aggagtttgg accgtgtctc agttccaatg tgggggacct 1140
tcctctcaga acccctatcc atcgaaggtt tggtgagccg ttacctcacc aactgcctaa 1200
tggaasgcat ccccatcgat aaccgaaatt ctttaataac aagaccatgc ggtctgatta 1260
taccatcggg tattaatctt tctttcgaaa ggctatcccc gagttatcgg caggttggat 1320
acgtgttact cacccgtgcg ccggtcgcca tctccagttt gcaagcaaac tgaaatgctg 1380
cccctcgact gca 1393
(Bacteroides thetaiotaomicron gene for 16S rRNA-BT-D)
SEQ ID NO: 7
gctccttgcg gttacgtact tcaggtaccc ccggctttca tggcttgacg ggcggtgtgt 60
acaaggcccg ggaacgtatt caccgcgcca tcgctgatgc gcgattacta gcgaatccag 120
cttcacgaag tcgggttgca gacttcgatc cgaactgaga gaggcttttg ggattagcat 180
cctgtcacca ggtagctgcc ttctgtaccc cccattgtaa cacgtgtgta gccccggacg 240
taagggccgt gctgatttga cgtcatcccc accttcctca catcttacga cggcagtctc 300
tctagagtcc tcagcatgac ctgttagtaa ctaaagataa gggttgcgct cgttatggca 360
cttaagccga cacctcacgg cacgagctga cgacaaccat gcagcacctt cacatttgcc 420
ttacggctat actgtttcca gtatattcaa atgcaattta agcccgggta aggttcctcg 480
cgtatcatcg aattaaacca catgttcctc cgcttgtgcg ggcccccgtc aattcctttg 540
agtttcaccg ttgccggcgt actccccagg tggaatactt aatgctttcg cttggccgct 600
tactgtatat cccaaacagc gagtattcat cctttactgt gtggactacc agggtatcta 660
atcctgtttg atacccacac tttcgagcat cagtgtcagt tgcagtccag tgagctgcct 720
tcgcaatcgg agttcttcgt gatatctaag catttcaccg ctacaccacg aattccgccc 780
acctctactg tactcaagac agccagtatc aactgcaatt ttacggttga gccgcaaact 840
ttcacaactg acttaactgt ccacctacgc tccctttaaa cccaataaat ccggataacg 900
ctcggatcct ccgtattacc gcggctgctg gcacggagtt agccgatcct tattcatatg 960
gtacatacaa aattccacac gtggaaaact ttattcccat ataaaagaag tttacaaccc 1020
atagggcagt catccttcac gctacttggc tggttcaggc ctgcgcccat tgaccaatat 1080
tcctcactgc tgcctcccgt aggagtttgg accgtgtctc agttccaatg tgggggacct 1140
tcctctcaga acccctatcc atcgaaggtt tggtgagccg ttacctcacc aactgcctaa 1200
tggaacgcat ccccatcgat aaccgaaatt ctttaataac aagaccatgc ggtctgatta 1260
taccatcggg tattaatctt tctttcgaaa ggctatcccc gagttatcgg caggtggata 1320
cgtgttactc acccgtgcgc cggtcgccat ctccagtttg caagcaaact gaaatgctgc 1380
ccctcgactg catg 1394
(Bacteroides thetaiotaomicron gene for 16S rRNA-BT-E)
SEQ ID NO: 8
gctccttgcg gttacgtact tcaggtaccc ccggctttca tggcttgacg ggcggtgtgt 60
acaaggcccg ggaacgtatt caccgcgcca tcgctgatgc gcgattacta gcgaatccag 120
cttcacgaag tcgggttgca gacttcgatc cgaactgaga gaggcttttg ggattagcat 180
cctgtcacca ggtagctgcc ttctgtaccc cccattgtaa cacgtgtgta gccccggacg 240
taagggccgt gctgatttga cgtcatcccc accttcctca catcttacga cggcagtctc 300
tctagagtcc tcagcatgac ctgttagtaa ctaaagataa gggttgcgct cgttatggca 360
cttaagccga cacctcacgg cacgagctga cgacaaccat gcagcacctt cacatttgcc 420
ttacggctat actgtttcca gtatattcaa atgcaattta agcccgggta aggttcctcg 480
cgtatcatcg aattaaacca catgttcctc cgcttgtgcg ggcccccgtc aattcctttg 540
agtttcaccg ttgccggcgt actccccagg tggaatactt aatgctttcg cttggccgct 600
tactgtatat cccaaacagc gagtattcat cgtttactgt gtggactacc agggtatcta 660
atcctgtttg atacccacac tttcgagcat cagtgtcagt tgcagtccag tgagctgcct 720
tcgcaatcgg agttcttcgt gatatctaag catttcaccg ctacaccacg aattccgccc 780
acctctactg tactcaagac agccagtatc aactgcaatt ttacggttga gccgcaaact 840
ttcacaactg acttaactgt ccacctacgc tccctttaaa cccaataaat ccggataacg 900
ctcggatcct ccgtattacc gcggctgctg gcacggagtt agccgatcct tattcatatg 960
gtacatacaa aattccacac gtggaaaact ttattcccat ataaaagaag tttacaaccc 1020
atagggcagt catccttcac gctacttggc tggttcaggc ctgcgcccat tgaccaatat 1080
tcctcactgc tgcctcccgt aggagtttgg accgtgtctc agttccaatg tgggggacct 1140
tcctctcaga acccctatcc atcgaaggtt tggtgagccg ttacctcacc aactgcctaa 1200
tggaacgcat ccccatcgat aaccgaaatt ctttaataac aagaccatgc ggtctgatta 1260
taccatcggg tattaatctt tctttcgaaa ggctatcccc gagttatcgg caggttggat 1320
acgtgttact cacccgtgcg ccggtcgcca tctccagttt gcaagcaaac tgaaatgctg 1380
cccctcgact gcatg 1395
(Bacteroides thetaiotaomicron gene for 16S rRNA-BT-F)
SEQ ID NO: 9
gctccttgcg gttacgtact tcaggtaccc ccggctttca tggcttgacg ggcggtgtgt 60
acaaggcccg ggaacgtatt caccgcgcca tggctgatgc gcgattacta gcgaatccag 120
cttcacgaag tcgggttgca gacttcgatc cgaactgaga gaggcttttg ggattagcat 180
cctgtcacca ggtagctgcc ttctgtaccc cccattgtaa cacgtgtgta gccccggacg 240
taagggccgt gctgatttga cgtcatcccc accttcctca catcttacga cggcagtctc 300
tctagagtcc tcagcatgac ctgttagtaa ctaaagataa gggttgcgct cgttatggca 360
cttaagccga cacctcacgg cacgagctga cgacaaccat gcagcacctt cacatttgcc 420
ttacggctat actgtttcca gtatattcaa atgcaattta agcccgggta aggttcctcg 480
cgtatcatcg aattaaacca catgttcctc cgcttgtgcg ggcccccgtc aattcctttg 540
agtttcaccg ttgccggcgt actccccagg tggaatactt aatgctttcg cttggccgct 600
tactgtatat cccaaacagc gagtattcat cgtttactgt gtggactacc agggtatcta 660
atcctgtttg atacccacac tttcgagcat cagtgtcagt tgcagtccag tgagctgcct 720
tcgcaatcgg agttcttcgt gatatctaag catttcaccg ctacaccacg aattccgccc 780
acctctactg tactcaagac agccagtatc aactgcaatt ttacggttga gccgcaaact 840
ttcacaactg acttaactgt ccacctacgc tccctttaaa cccaataaat ccggataacg 900
ctcggatcct ccgtattacc gcggctgctg gcacggagtt agccgatcct tattcatatg 960
gtacatacaa aattccacac gtggaaaact ttattcccat ataaaagaag tttacaaccc 1020
atagggcagt catccttcac gctacttggc tggttcaggc ctgcgcccat tgaccaatat 1080
tcctcactgc tgcctcccgt aggagtttgg accgtgtctc agttccaatg tgggggacct 1140
tcctctcaga acccctatcc atcgaaggtt tggtgagccg ttacctcacc aactgcctaa 1200
tggaacgcat ccccatcgat aaccgaaatt ctttaataac aagaccatgc ggtctgatta 1260
taccatcggg tattaatctt tctttcgaaa ggctatcccc gagttatcgg caggtaggat 1320
acgtgttact cacccgtgcg ccggtcgcca tctccagttt gcaagcaaac tgaaatgctg 1380
cccctcgact gcatg 1395
(Bacteroides thetaiotaomicron gene for 16S rRNA-BT-G)
SEQ ID NO: 10
tttactagga cgctcttgcg gttacgtact tcaggtaccc ccggctttca tggcttgacg 60
ggcggtgtgt acaaggcccg ggaacgtatt caccgcgcca tggctgatgc gcgattacta 120
gcgaatccag cttcacgaag tcgggttgca gacttcgatc cgaactgaga gaggcttttg 180
ggattagcat cctgtcacca ggtagctgcc ttctgtaccc cccattgtaa cacgtgtgta 240
gccccggacg taagggccgt gctgatttga cgtcatcccc accttcctca catcttacga 300
cggcagtctc tctagagtcc tcagcatgac ctgttagtaa ctaaagataa gggttgcgct 360
cgttatggca cttaagccga cacctcacgg cacgagctga cgacaaccat gcagcacctt 420
cacatttgcc ttacggctat actgtttcca gtatattcaa atgcaattta agcccgggta 480
aggttcctcg cgtatcatcg aattaaacca catgttcctc cgcttgtgcg ggcccccgtc 540
aattcctttg agtttcaccg ttgccggcgt actccccagg tggaatactt aatgctttcg 600
cttggccgct tactgtatat cccaaacagc gagtattcat cgtttactgt gtggactacc 660
agggtatcta atcctgtttg atacccacac tttcgagcat cagtgtcagt tgcagtccag 720
tgagctgcct tcgcaatcgg agttcttcgt gatatctaag catttcaccg ctacaccacg 780
aattccgccc acctctactg tactcaagac agccagtatc aactgcaatt ttacggttga 840
gccgcaaact ttcacaactg acttaactgt ccacctacgc tccctttaaa cccaataaat 900
ccggataacg ctcggatcct ccgtattacc gcggctgctg gcacggagtt agccgatcct 960
tattcatatg gtacatacaa aattccacac gtggaaaact ttattcccat ataaaagaag 1020
tttacaaccc atagggcagt catccttcac gctacttggc tggttcaggc ctgcgcccat 1080
tgaccaatat tcctcactgc tgcctcccgt aggagtttgg accgtgtctc agttccaatg 1140
tgggggacct tcctctcaga acccctatcc atcgaaggtt tggtgagccg ttacctcacc 1200
aactgcctaa tggaacgcat ccccatcgat aaccgaaatt ctttaataac aagaccatgc 1260
ggtctgatta taccatcggg tattaatctt tctttcgaaa ggctatcccc gagttatcgg 1320
caggttggat acgtgttact cacccgtgcg ccggtcgcca tctccagttg caagcaaact 1380
gaaatgctgc ccctcgactg catgtgtagc cg 1412
(Bacteroides thetaiotaomicron gene for 16S rRNA-BT-H)
SEQ ID NO: 11
ggacgctcct tgcggttacg tacttcaggt acccccggct ttcatggctt gacgggcggt 60
gtgtacaagg cccgggaacg tattcaccgc gccatggctg atgcgcgatt actagcgaat 120
ccagcttcac gaagtcgggt tgcagacttc gatccgaact gagagaggct tttgggatta 180
gcatcctgtc accaggtagc tgccttctgt accccccatt gtaacacgtg tgtagccccg 240
gacgtaaggg ccgtgctgat ttgacgtcat ccccaccttc ctcacatctt acgacggcag 300
tctctctaga gtcctcagca tgacctgtta gtaactaaag ataagggttg cgctcgttat 360
ggcacttaag ccgacacctc acggcacgag ctgacgacaa ccatgcagca ccttcacatt 420
tgccttacgg ctatactgtt tccagtatat tcaaatgcaa tttaagcccg ggtaaggttc 480
ctcgcgtatc atcgaattaa accacatgtt cctccgcttg tgcgggcccc cgtcaattcc 540
tttgagtttc accgttgccg gcgtactccc caggtggaat acttaatgct ttcgcttggc 600
cgcttactgt atatcgcaaa cagcgagtat tcatcgttta ctgtgtggac taccagggta 660
tctaatcctg tttgataccc acactttcga gcatcagtgt cagttgcagt ccagtgagct 720
gccttcgcaa tcggagttct tcgtgatatc taagcatttc accgctacac cacgaattcc 780
gcccacctct actgtactca agacagccag tatcaactgc aattttacgg ttgagccgca 840
aactttcaca actgacttaa ctgtccacct acgctccctt taaacccaat aaatccggat 900
aacgctcgga tcctccgtat taccgcggct gctggncacg gagttagccg atccttattc 960
atatggtaca tacaaaattc cacacgtgga aaactttatt cccatataaa agaagtttac 1020
aacccatagg gcagtcatcc ttcacgctac ttggctggtt caggcctgcg cccattgacc 1080
aatattcctc actgctgcct cccgtaggag tttggaccgt gtctcagttc caatgtgggg 1140
gaccttcctc tcagaacccc tatccatcga aggtttggtg agccgttacc tcaccaactg 1200
cctaatggaa cgcatcccca tcgataaccg aaattcttta ataacaagac catgcggtct 1260
gattatacca tcgggtatta atctttcttt cgaaaggcta tccccgagtt atcggcaggt 1320
tggatacgtg ttactcaccc gtgcgccggt cgccatctcc agtttgcaag caaactgaaa 1380
tgctgcccct cgactgca 1398
(Bacteroides thetaiotaomicron gene for 16S rRNA-BT-I)
SEQ ID NO: 12
gctccttgcg gttacgtact tcaggtaccc ccggctttca tggcttgacg ggcggtgtgt 60
acaaggcccg ggaacgtatt caccgcgcca tggctgatgc gcgattacta gcgaatccag 120
cttcacgaag tcgggttgca gacttcgatc cgaactgaga gaggcttttg ggattagcat 180
cctgtcacca ggtagctgcc ttctgtaccc cccattgtaa cacgtgtgta gccccggacg 240
taagggccgt gctgatttga cgtcatcccc accttcctca catcttacga cggcagtctc 300
tctagagtcc tcagcatgac ctgttagtaa ctaaagataa gggttgcgct cgttatggca 360
cttaagccga cacctcacgg cacgagctga cgacaaccat gcagcacctt cacatttgcc 420
ttgcggctaa cctgtttcca gawtattcaa atgcaattta agcccgggta aggttcctcg 480
cgtatcatcg aattaaacca catgttcctc cgcttgtgcg ggcccccgtc aattcctttg 540
agtttcaccg ttgccggcgt actccccagg tggaatactt aatgctttcg cttggccgct 600
tactgtatat cccaaacagc gagtattcat cctttactgt gtggactacc agggtatcta 660
atcctgtttg atacccacac tttcgagcat cagtgtcagt tgcagtccag tgagctgcct 720
tcgcaatcgg agttcttcgt gatatctaag catttcaccg ctacaccacg aattccgccc 780
acctctactg tactcaagac agccagtatc aactgcaatt ttacggttga gccgcaaact 840
ttcacaactg acttaactgt ccacctacgc tccctttaaa cccaataaat ccggataacg 900
ctcggatcct ccgtattacc gcggctgctg gcacggagtt agccgatcct tattcatatg 960
gtacatacaa aattccacac gtggaaaact ttattcccat ataaaagaag tttacaaccc 1020
atagggcagt catccttcac gctacttggc tggttcaggc tttcgtccat tgaccaatat 1080
tcctcactgc tgcctcccgt aggagtttgg accgtgtctc agttccaatg tgggggacct 1140
tcctctcaga acccctatcc atcgaaggtt tggtgagccg ttacctcacc aactgcctaa 1200
tggaacgcat ccccatcgat aaccgaaatt ctttaataac aagaccatgc ggtctaatta 1260
taccatcggg tattaatctt tctttcgaaa ggctatcccc gagttatcgg caggttggat 1320
acgtgttact cacccgtgcg ccggtcgcca tctccagttt gcaagcaaac tgaaatgctg 1380
cccctcgact gca 1393

### REFERENCES

[1] Spor et al. (2011) Nat Rev Microbiol. 9(4):279-90.
[2] Tap et al. (2009), Environ Microbiol, 11(10):2574-84
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15; 122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] WO 2013/050792
[11] WO 03/046580
[12] WO 2013/008039
[13] WO 2014/167338
[14] Lee and Lee (2014) World J Gastroenterol. 20(27): 8886-8897.
[15] Xie et al. (2016) Journal Dairy Sci. 99:6913-6921
[16] Y Q et al. (2016), J. Dig. Dis., “Therapeutic Modulation of the Gut Microbiota in IBD—More Questions to Be Answered”, Oct. 15, 1751-2980, 12422, Epub ahead of print.
[17] Gong et al. Gastroenterol Res Pract. 2016; 2016:6951091
[18] Ott and Schreiber Gut. 2006 August; 55(8): 1207.
[19] Lozupone (2012). Nature. 2012 Sep. 13; 489 (7415): 220-230
[20] Claesson, et al. (2012) Nature, 488, 178-184.
[21] Hansen, et al., (2010), Curr. Opin. Gastroenterol., 26(6): 564-571.
[22] Turnbaugh et al. Nature, 457(7228): 480-484.
[23] Mosca et al. (2016) Front. Microbiol. 7:455
[24] Faith et al. (2013), Science, 341(6141): 1237439
[25] Wang et al. (2009) ISME J. 3(8): 944-954.
[26] Faith et al. (2013), Science, 341(6141): 1237439
[27] Masco et al. (2003) Systematic and Applied Microbiology, 26:557-563.
[28] Srůtková et al. (2011) J. Microbiol. Methods, 87(1): 10-6.
[29] Miyamoto-Shinohara et al. (2008) J. Gen. Appl. Microbiol., 54, 9-24.
[30] Cryopreservation and Freeze-Drying Protocols, ed. by Day and McLellan, Humana Press.
[31] Leslie et al. (1995) Appl. Environ. Microbiol. 61, 3592-3597.
[32] Mitropoulou et al. (2013)J Nutr Metab. (2013) 716861.
[33] Kailasapathy et al. (2002) Curr Issues Intest Microbiol. 3(2):39-48.
[34] Handbook of Pharmaceutical Excipients, 2nd Edition, (1994), Edited by A Wade and P J Weller
[35] Remington's Pharmaceutical Sciences, Mack Publishing Co. (A. R. Gennaro edit. 1985)
[36] US 2016/0067188
[37] Handbook of Microbiological Media, Fourth Edition (2010) Ronald Atlas, CRC Press.
[38] Maintaining Cultures for Biotechnology and Industry (1996) Jennie C. Hunter-Cevera, Academic Press
[39] Strobel (2009) Methods Mol Biol. 581:247-61.
[40] Gennaro (2000) Remington: The Science and Practice of Pharmacy. 20th edition, ISBN: 0683306472.
[41] Molecular Biology Techniques: An Intensive Laboratory Course, (Ream et al., eds., 1998, Academic Press).
[42] Methods In Enzymology (S. Colowick and N. Kaplan, eds., Academic Press, Inc.)
[43] Handbook of Experimental Immunology, Vols. I-IV (D. M. Weir and C. C. Blackwell, eds, 1986, Blackwell Scientific Publications)
[44] Sambrook et al. (2001) Molecular Cloning: A Laboratory Manual, 3rd edition (Cold Spring Harbor Laboratory Press).
[45] Handbook of Surface and Colloidal Chemistry (Birdi, K. S. ed., CRC Press, 1997)
[46] Ausubel et al. (eds) (2002) Short protocols in molecular biology, 5th edition (Current Protocols).
[47] PCR (Introduction to Biotechniques Series), 2nd ed. (Newton & Graham eds., 1997, Springer Verlag)
[48] Current Protocols in Molecular Biology (F. M. Ausubel et al., eds., 1987) Supplement 30
[49] Smith & Waterman (1981) Adv. Appl. Math. 2: 482-489.

1. A composition comprising a bacterial strain of the species Bacteroides thetaiotaomicron, for use in a method of increasing the microbiota diversity and/or inducing stability of the microbiota of a subject.

2.-39. (canceled)
